(19) United States
(12) Patent Application Publication
(10) Pub. No.: US 2024/0091284 A1
(43) Pub. Date: Mar. 21, 2024

(54) ONCOLYTIC VIRUS COMPRISING IMMUNOMODULATORY TRANSGENES AND USES THEREOF

(71) Applicant: Arizona Board of Regents on Behalf of Arizona State University, Scottsdale, AZ (US)

(72) Inventors: Douglas Grant McFADDEN, Tempe, AZ (US); Lino TORRES-DOMINGUEZ, Tempe, AZ (US); Nancy VILLA, Tempe, AZ (US); Mohammed Masmudur RAHMAN, Chandler, AZ (US)

(21) Appl. No.: 17/767,857

(22) PCT Filed: Oct. 9, 2020
(86) PCT No.: PCT/US2020/055083

§ 371 Date: Apr. 8, 2022

Related U.S. Application Data
(60) Provisional application No. 62/913,658, filed on Oct. 10, 2019.

(51) Int. Cl. A61K 35/768; A61K 35/15; A61P 33/00

(52) U.S. Cl. CPC A61K 35/768; A61K 35/15; A61P 33/00

(57) ABSTRACT

The disclosure provides Myxoma virus that expresses one or more immunomodulatory transgenes and its use in inhibiting and/or treating a hematological cancer in a subject. The disclosure also provides a leukocyte having a Myxoma virus that expresses one or more immunomodulatory transgenes and the use of the leukocyte for inhibiting and/or treating a hematological cancer in a subject.

### CROSS REFERENCE

This application claims the benefit of U.S. Provisional Patent Application No. 62/913,658, filed Oct. 10, 2019, which is incorporated herein by reference in its entirety.

### TECHNICAL FIELD

This disclosure relates to myxoma viruses and their uses for treatment of cancers, for example, treatment of hematological cancers with a myxoma virus that expresses one or more immunomodulatory transgenes.

### BACKGROUND

Current treatments used to treat various types of cancer tend to work by poisoning or killing the cancerous cell. Unfortunately, treatments that are toxic to cancer cells typically tend to be toxic to healthy cells as well. Moreover, effective treatments for cancer remain elusive. Current mainstream therapies such as chemotherapy and radiotherapy can have a narrow therapeutic window (e.g., a concentration high enough to achieve efficacy but low enough to avoid toxicity). These types of therapies are considered blunt tools that have limited applicability due to the varying types of tumor cells and the limited window in which these treatments can be administered.

### SUMMARY

Disclosed herein, in some aspects is an engineered myxoma virus (MYXV) comprising a transgene encoding one or more immunomodulatory proteins.

In some embodiments, the one or more immunomodulatory proteins is Stimulator of interferon genes (STING), interleukin-12 (IL-12), Fusion-associated small transmembrane (FAST), an immune checkpoint inhibitor, a tumor necrosis factor (TNF) protein, or a combination thereof. In some embodiments, the IL-12 is an IL-12A. In some embodiments, the IL-12 is an IL-12B. In some embodiments, the immune checkpoint inhibitor is a PD-L1 binding molecule. In some embodiments, the PD-L1 binding molecule is an anti-PD-L1 antibody or an antigen-binding fragment thereof. In some embodiments, the one or more immunomodulatory proteins is capable of stimulating a toll like receptor (TLR), activating Nuclear factor-κB (NF-κB), or activating an interferon regulatory factor (IRF). In some embodiments, the engineered MYXV comprises a modification at or adjacent to one or more genes selected from the group consisting of M001R, M002R, M003.1R, M003.2R, M004.1R, M004R, M005R, M006R, M007R, M008.1R, M008R, M009L, M013, M036L, M063L, M11L, M128L, M131R, M135R, M136R, M141R, M148R, M151R, M152R, M153R, M154L, M156R, M-T2, M-T4, M-T5, M-T7, and SOD. In some embodiments, the engineered MYXV comprises a modification at or adjacent to M011L, M063, M135R, M136R, M-T2, M-T4, M-T5, M-T7 or SOD. In some embodiments, the modification comprises a deletion or an insertion. In some embodiments, the transgene replaces a portion of M011L, M063, M135R, M136R, M-T2, M-T4, M-T5, M-T7, or SOD. In some embodiments, the transgene is located between the M135R and M136R genes of the genome of the MYXV. In some embodiments, the transgene replaces a portion of M135R. In some embodiments, the engineered MYXV is an M135R knockout. In some embodiments, the engineered MYXV is a SOD knockout. In some embodiments, the engineered MYXV further comprises a reporter gene. In some embodiments, the reporter gene encodes a fluorescent protein, a luminescent substrate or an enzyme. In some embodiments, the engineered MYXV increases autophagy in infected cells by at least 5% compared to a MYXV that lacks the transgene as determined by an LC3-I to LC3-II conversion assay. In some embodiments, the engineered MYXV increases killing of infected cancer cells by at least 5% compared to a MYXV that lacks the transgene as determined by an in vitro flow cytometric assay. In some embodiments, the engineered MYXV increases killing of uninfected cancer cells by at least 5% compared to a MYXV that lacks the transgene as determined by an in vitro flow cytometric assay. In some embodiments, the engineered MYXV is present in a pharmaceutical composition that comprises the engineered MYXV and a pharmaceutically-acceptable excipient. In some embodiments, the pharmaceutical composition is formulated for systemic administration. In some embodiments, the pharmaceutical composition is formulated for local administration. In some embodiments, the pharmaceutical composition is formulated for parenteral administration. In some embodiments, a plurality of cells is exposed ex vivo to the engineered MYXV, wherein the plurality of cells comprises a peripheral blood mononuclear cell (PBMC), a bone marrow (BM) cell, or a combination thereof. In some embodiments, the plurality of cells is derived from a single subject. In some embodiments, the engineered MYXV, the pharmaceutical composition, or the plurality of cells is administered to a subject in need thereof in a method of treating cancer. In some embodiments, the engineered MYXV is adsorbed ex vivo onto the surface of at least a portion of the plurality of cells. In some embodiments, the engineered MYXV is adsorbed by exposing the plurality of cells to the engineered MYXV under a condition that permits binding of the engineered MYXV to a surface of the plurality of cells. In some embodiments, the engineered MYXV is infected to at least a portion of the plurality of cells. In some embodiments, the cancer is a solid tumor. In some embodiments, the cancer has metastasized to a second location in the subject. In some embodiments, the second location comprises a lung, a brain, a liver and/or a lymph node of the subject. In some embodiments, the cancer comprises osteosarcoma, triple negative breast cancer, or melanoma. In some embodiments, an additional therapeutic agent is administered to the subject. In some embodiments, the additional therapeutic agent is administered to the subject prior to administering the composition. In some embodiments, the additional therapeutic agent is administered to the subject after administering the composition. In some embodiments, the additional therapeutic agent is co-administered to the subject with the composition. In some embodiments, the subject is a human. In some embodiments, the subject has or is suspected of having a cancer. In some embodiments, the engineered MYXV is capable of infecting cells that have a deficient innate anti-viral response. In some embodiments, the cells that have a deficient innate anti-viral response comprise cancer cells. In some embodiments, the plurality of cells is obtained or derived from the subject's tissue. In some embodiments, the plurality of cells are from a donor that is allogeneic to the subject. In some embodiments, the plurality of cells is obtained or derived from a donor's tissue that is HLA-matched, HLA-mismatched, haploidentical, or a combination thereof relative to the subject. In some embodiments, the engineered MYXV, the pharmaceutical composition, or the composition is part of a kit.

Some embodiments relate to a myxoma virus (MYXV) engineered to express one or more immunomodulatory proteins.

Some embodiments relate to a myxoma virus (MYXV) engineered to express one or more immunomodulatory proteins capable of stimulating the toll like receptors (TLR) or activating Nuclear factor-κB (NF-κB) or (interferon regulatory factor) IRF.

Some embodiments relate to a pharmaceutical composition comprising a myxoma virus described herein and a pharmaceutically acceptable excipient.

Some embodiments relate to a composition comprising peripheral blood mononuclear cells (PBMCs), bone marrow (BM) cells, or a combination thereof treated ex vivo by a myxoma virus (MYXV) engineered to express an immunomodulatory protein.

Some embodiments relate to a method of inhibiting or treating a cancer in a subject in need thereof, comprising administering to the subject a myxoma virus (MYXV) engineered to express an immunomodulatory protein.

Some embodiments relate to a method of inhibiting or treating a cancer in a subject in need thereof, comprising administering to the subject a composition described herein.

Some embodiments relate to a kit comprising a myxoma virus or a pharmaceutical composition described herein.

### BRIEF DESCRIPTION OF THE DRAWINGS

The novel features of certain embodiments of the disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1 provides an example schematic of a recombination plasmid design that can be used to generate a recombinant MYXV of the disclosure comprising an immunomodulatory transgene.

FIG. 2 illustrates the design of a recombination plasmid used to generate an MYXV of the disclosure comprising a STING transgene, and the modified MYXV genome after recombination

FIG. 3A illustrates detection of STING via agarose gel electrophoresis of PCR products obtained from screening candidate MYXV-STING clones or controls.

FIG. 3B illustrates detection of STING via western blot of lysates from RK13 cells infected with candidate MYXV-STING clone or controls.

FIG. 4 demonstrates the replication capacity of MYXV-STING was similar to a control virus in RK13 cells.

FIG. 5A, FIG. 5B, and FIG. 5C are microscopy images obtained from RK13, Vero, and A549 cell lines that were mock-infected (FIG. 5A), infected with MYXV-M135KO-GFP (FIG. 5B), or infected with MYXV-M135KO-GFP-STING (FIG. 5C).

FIG. 6 illustrates the presence of LC3-II detected by Western Blot of lysates from A549 cells that were mock-infected, infected with MYXV-STING, or infected with MYXV-GFP.

FIG. 7A and FIG. 7B demonstrate infection of THP-1 cells by MYXV of the disclosure at 24 and 48 hours post-infection, respectively.

FIG. 8A and FIG. 8B demonstrate infection of U266 cells by MYXV of the disclosure at 24 and 48 hours post-infection, respectively.

FIG. 9 demonstrates killing of THP-1 cells by MYXV of the disclosure, evaluated by flow cytometry.

FIG. 10 demonstrates killing of U266 cells by MYXV of the disclosure, evaluated by flow cytometry.

FIG. 11 demonstrates killing of primary human multiple myeloma cells by MYXV of the disclosure, evaluated by flow cytometry.

FIG. 12 demonstrates loss of Pacific Blue 420-labeled MM population shown from the same primary cell sample, analyzed 6 and 24 hours post BM extraction during bone marrow biopsy.

FIGS. 13A-13C show the BOR-resistant VK12598 cell line is susceptible to MYXV. FIG. 13A shows binding of MYXV (Venus+) to the VK12598 cell line. FIG. 13B shows productive infection of the VK12598 cell line via fluorescence microscopy. FIG. 13C shows productive infection of the VK12598 cell line via flow cytometry.

FIGS. 14A and 14B show MYXV binding and infection of the multi-drug resistant VK12653 cell line. FIG. 14A shows binding of MYXV (Venus+) to the VK12653 cell line. FIG. 14B shows productive infection of the VK12598 cell line via fluorescence microscopy and flow cytometry.

FIGS. 15A-15C show ex vivo therapy with myxoma virus to treat pre-existing multiple myeloma cancer in auto-transplant recipients. FIG. 15A shows a Western Blot providing the M-spike of mice four weeks post implantation with VK12598 cells (top panel) and four experimental cohorts (bottom panel). Levels of M-spike four weeks post-MM cell implantation.

FIG. 15B shows the percentage of MM cells (CD138+B220−) in a representative mock-treated mouse with low M-spike (0.1) and the percentage of MM (CD138+B220−) in a representative bone marrow-recipient mouse with high M-spike (0.6). FIG. 15C shows the M-spike of a mouse treated with bone marrow that had been ex vivo treated with MYXV-M135KO-GFP, with no M-spike band detected on day 8, day 29, and day 37 post-transplant.

FIG. 16A shows the percent of THP-1 cells that were GFP positive at 24 and 48 hours post-infection with MYXV-WT-GFP, MYXV-M135KO-GFP, MYXV-SODKO-TNFα-GFP, MYXV-anti-muPDL1-GFP, or MYXV-p14FAST-GFP.

FIG. 16B shows the percent of U266 cells that were GFP positive at 24 and 48 hours post-infection with MYXV-WT-GFP, MYXV-M135KO-GFP, MYXV-SODKO-TNFα-GFP, MYXV-anti-muPDL1-GFP, or MYXV-p14FAST-GFP.

FIG. 17A illustrates the percent of infected THP-1 cells that were killed at 24 and 48 hours by MYXV-WT-GFP, MYXV-SODKO-TNFα-GFP, and MYXV-p14FAST-GFP.

FIG. 17B illustrates the percent of uninfected THP-1 cells that were killed at 24 and 48 hours by MYXV-WT-GFP, MYXV-SODKO-TNFα-GFP, and MYXV-p14FAST-GFP.

FIG. 18A illustrates the percent of infected U266 cells that were killed at 24 and 48 hours by MYXV-WT-GFP and MYXV-p14FAST-GFP.

FIG. 18B illustrates the percent of uninfected U266 cells that were killed at 24 and 48 hours by MYXV-WT-GFP and MYXV-p14FAST-GFP.

FIG. 19 provides the ratio of dead THP-1 cells to infected THP-1 cells for cultures infected with MYXV-WT-GFP, MYXV-M135KO-GFP, MYXV-SODKO-TNFα-GFP, MYXV-anti-muPDL1-GFP, or MYXV-p14FAST-GFP.

FIG. 20 provides the ratio of dead U266 cells to infected U266 cells, for cultures infected with MYXV-WT-GFP, MYXV-M135KO-GFP, MYXV-SODKO-TNFα-GFP, MYXV-anti-muPDL1-GFP, or MYXV-p14FAST-GFP.

FIG. 21 shows the percent of primary human CD138+ multiple myeloma cells that were infected by (GFP positive) MYXV-WT-GFP, MYXV-M135KO-GFP, and MYXV-SODKO-TNFα-GFP.

### DETAILED DESCRIPTION

Aspects of this disclosure relate to oncolytic virus recombinant constructs expressing immunomodulatory transgenes and their uses for treating cancer such as hematologic cancer. The oncolytic virus can be a Myxoma virus (MYXV), and the immunomodulatory transgenes used in the construct can include Stimulator of interferon genes (STING), antibodies or antigen-binding fragments thereof that bind to programmed death ligand 1 (anti-PDL1), Fusion-associated small transmembrane (FAST), tumor necrosis factor alpha (TNFα), and interleukin 12 (IL-12), or a combination thereof. The MYXV described herein can be used to treat hematological cancers, including minimal residual disease (MRD) and drug-resistant MRD.

The MYXV described herein can be a more effective therapy to treat hematologic cancer such as relapsed Multiple Myeloma (MM) disease, and to fully eliminate the refractory and drug-resistant MRD. MM is a hematologic malignancy that can be characterized by a clonal expansion of malignant plasma cells resulting in end organ damage, including lytic bone lesions, anemia, renal failure, or hypercalcemia (Hari P. Recent advances in understanding multiple myeloma. Hematol Oncol Stem Cell Ther. 2017; In press). The bone marrow (BM) tumor microenvironment of MM plays a key role supporting and sustaining the differentiation, migration, proliferation, survival, and drug resistance of malignant MM cells (Kawano Y, Moschetta M, Manier S, Glavey S, Görgün GT, Roccaro A M, et al. Targeting the bone marrow microenvironment in multiple myeloma. Immunol Rev. 2015; 263(1)). Autologous stem cell transplantation for transplant eligible patients, along with chemotherapy, can be a standard treatment for MM (Landgren O, Lu S X, and Hultcrantz M. MRD Testing in Multiple Myeloma: The Main Future Driver for Modern Tailored Treatment. Semin Hematol. 2018; 55(1):44-50; Hoyos V, and I. B. The immunotherapy era of myeloma: monoclonal antibodies, vaccines, and adoptive T-cell therapies. Blood. 2016; 128(13):1679-87). However, a major hurdle of these therapies can be the relapse of the disease due to neoplastic clones that can serve as a reservoir of therapy-resistant MM cells, resulting in minimal residual disease (MRD).

Despite improvement in outcomes, MM is still considered incurable for most patients, and poor survival rates are observed in those patients with high-risk features (Bustoros M, Mouhieddine T H, Detappe A, and IM. G. Established and Novel Prognostic Biomarkers in Multiple Myeloma. Am Soc Clin Oncol Educ Book. 2017; 37:548-60). Oncolytic viruses such as MYXV are mammalian viruses that can be designed and/or selected for their ability to selectively infect and kill transformed cancer cells, and for their ability to activate the host immune system. The MYXV described herein utilizes immunomodulatory transgenes and can work in combination with the host immune systems to target cancer cells. Therefore, the Myxoma virus described herein can help reduce or eliminate the refractory and drug-resistant minimal residual disease and can be more effective to treat relapsed MM disease.

### Definitions

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCR Publishers, Inc., 1995 (ISBN 1-56081-569-8).

Unless otherwise explained, all technical and scientific terms used herein have the same meaning as commonly understood by one of ordinary skill in the art to which this disclosure belongs. Although methods and materials similar or equivalent to those described herein can be used in the practice or testing of this disclosure, suitable methods and materials are described below. In addition, the materials, methods, and examples are illustrative only and not intended to be limiting.

The following explanations of terms and methods are provided to better describe the present compounds, compositions, and methods, and to guide those of ordinary skill in the art in the practice of the present disclosure. It is also to be understood that the terminology used in the disclosure is for the purpose of describing particular embodiments and examples only and is not intended to be limited.

As used herein, the singular forms “a,” “an,” and “the” are intended to include the plural forms as well, unless the context clearly indicates otherwise.

As used herein, the term “and/or” refers to and encompasses any and all possible combinations of one or more of the associated listed items, as well as the lack of combinations when interpreted in the alternative (“or”).

As used herein, “one or more” or at least one can mean one, two, three, four, five, six, seven, eight, nine, ten or more, up to any number.

As used herein, the term “comprises” or “comprising” mean “includes.” Hence “comprising A or B” means including A, B, or A and B. “Comprise” and variations of the term, such as “comprising”, “comprises” and “comprised”, as used herein, mean that various additional components or steps can be conjointly employed.

An “effective amount” or “therapeutically effective amount” refers to an amount of a compound or composition of this disclosure that is sufficient to produce a desired effect, which can be a therapeutic and/or beneficial effect. In this example, the effective amount can vary with the age, general condition of the subject, the severity of the condition being treated, the particular agent administered, the duration of the treatment, the nature of any concurrent treatment, the pharmaceutically acceptable carrier used, and like factors within the knowledge and expertise of skilled workers. As appropriate, an effective amount or therapeutically effective amount in any individual case can be determined by reference to the pertinent texts and literature and/or by experimentation. (See, for example, Remington, The Science and Practice of Pharmacy (latest edition)).

As used herein, the term “subject” and “patient” are used interchangeably and refer to both human and nonhuman animals. The term “nonhuman animals” of the disclosure includes all vertebrates, e.g., mammals and non-mammals, such as nonhuman primates, sheep, dog, cat, horse, cow, rodents (e.g., mice, rats, etc.) and the like. A subject can be a human. A subject can be a human patient. In some embodiments, the subject of this disclosure is a human subject.

The term “a cell” as used herein includes a single cell as well as a plurality or population of cells. Administering or exposing an agent to a cell can include in vitro, ex vivo, and in vivo administering or exposing.

A “subject in need thereof” or “a subject in need of” is a subject known to have, or that is suspected of having a cancer, such as a hematological cancer.

As used herein, the term “cancer” refers to a malignant neoplasm, for example, a neoplasm that has undergone characteristic anaplasia with loss of differentiation, increased rate of growth, invasion of surrounding tissue, and is capable of metastasis.

Residual cancer is cancer that remains in a subject after any form of treatment given to the subject to reduce or eradicate cancer. Metastatic cancer is a cancer at one or more sites in the body, e.g., a second site, other than the site of origin of the original (primary) cancer from which the metastatic cancer is derived. Local recurrence is reoccurrence of the cancer at or near the same site (such as in the same tissue) as the original cancer. Hematologic cancer is a cancer that affects the blood, bone marrow, and/or lymphatic system.

Non-limiting examples of hematologic cancers include leukemia, lymphoma, and myeloma, such as: multiple myeloma (MM); active multiple myeloma; smoldering multiple myeloma; plasmacytoma; solitary plasmacytoma of the bone; extramedullary plasmacytoma; light chain myeloma; non-secretory myeloma; immunoglobulin G (IgG) myeloma; immunoglobulin A (IgA) myeloma; immunoglobulin M (IgM) myeloma; immunoglobulin D (IgD) myeloma; immunoglobulin E (IgE) myeloma; hyperdiploid multiple myeloma; non-hyperdiploid multiple myeloma; Hodgkin lymphoma; non-Hodgkin lymphoma; acute lymphoblastic leukemia; acute myeloid leukemia; essential thrombocythemia; polycythemia vera; primary myelofibrosis; systemic mastocytosis; chronic myeloid leukemia; chronic neutrophilic leukemia; chronic eosinophilic leukemia; refractory anemia with ringed sideroblasts; refractory cytopenia with multilineage dysplasia; refractory anemia with excess blasts type 1; refractory anemia with excess blasts type 2; myelodysplastic syndrome (MDS) with isolated del (5q); MDS unclassifiable; chronic myelomonocytic leukemia (CML); atypical chronic myeloid leukemia; juvenile myelomonocytic leukemia; myeloproliferative/myelodysplastic syndromes—unclassifiable; B lymphoblastic leukemia/lymphoma; T lymphoblastic leukemia/lymphoma; diffuse large B-cell lymphoma; primary central nervous system lymphoma; primary mediastinal B-cell lymphoma; Burkitt lymphoma/leukemia; follicular lymphoma; chronic lymphocytic leukemia (CLL)/small lymphocytic lymphoma; B-cell prolymphocytic leukemia; lymphoplasmacytic lymphoma/Waldenstrom macroglobulinemia; Mantle cell lymphoma; marginal zone lymphomas; post-transplant lymphoproliferative disorders; HIV-associated lymphomas; primary effusion lymphoma; intravascular large B-cell lymphoma; primary cutaneous B-cell lymphoma; hairy cell leukemia; monoclonal gammopathy of unknown significance; Anaplastic large cell lymphoma, Angioimmunoblastic T-cell lymphoma, Hepatosplenic T-cell lymphoma, B-cell lymphoma, reticuloendotheliosis, reticulosis, Mucosa-associated lymphatic tissue lymphoma, B-cell chronic lymphocytic leukemia, Waldenstrom's macroglobulinemia, Lymphomatoid granulomatosis, Nodular lymphocyte predominant Hodgkin's lymphoma, plasma cell leukemia, Acute erythraemia and erythroleukaemia, Acute erythremic myelosis, Acute erythroid leukemia, Heilmeyer-Schoner disease, Acute megakaryoblastic leukemia, Mast cell leukemia, Panmyelosis, Acute panmyelosis with myelofibrosis, Lymphosarcoma cell leukemia, Stem cell leukemia, Chronic leukaemia of unspecified cell type, Subacute leukaemia of unspecified cell type, Accelerated phase chronic myelogenous leukemia, Acute promyelocytic leukemia, Acute basophilic leukemia, Acute eosinophilic leukemia, Acute monocytic leukemia, Acute myeloblastic leukemia with maturation, Acute myeloid dendritic cell leukemia, Adult T-cell leukemia/lymphoma, Aggressive NK-cell leukemia, B-cell chronic lymphocytic leukemia, B-cell leukemia, Chronic myelogenous leukemia, Chronic idiopathic myelofibrosis, Kahler's disease, Myelomatosis, Solitary myeloma, Plasma cell leukemia, Angiocentric immunoproliferative lesion, Lymphoid granulomatosis, Angioimmunoblastic lymphadenopathy, T-gamma lymphoproliferative disease, Waldenstrom's macroglobulinaemia, Alpha heavy chain disease, Gamma heavy chain disease, and Franklin's disease. In some embodiments, the hematological cancer is multiple myeloma.

As used herein, the term “chemotherapeutic agent” refers to any chemical agent with therapeutic usefulness in the treatment of diseases characterized by abnormal cell growth. Such diseases can include tumors, neoplasms, and cancer, as well as diseases characterized by hyperplastic growth such as psoriasis. In some embodiments, a chemotherapeutic agent is an agent of use in treating cancer, such as an anti-neoplastic agent. In some embodiments, a chemotherapeutic agent is a radioactive compound. One of skill in the art can readily identify a chemotherapeutic agent of use (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993). Combination therapy is the administration of more than one agent to treat cancer. For example, a myxoma virus expressing an immunomodulatory transgene can be administered, and one or more chemotherapeutic agents can be administered, simultaneously or separated in time in any order.

“Treat,” “treatment,” or “treating,” as used herein refers to administering a pharmaceutical composition to a patient suffering from a disease or condition. As used herein, the term “inhibiting or treating a disease,” such as cancer, refers to delaying or inhibiting the development or progression of a disease or condition. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, such a metastasis, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology, for example metastatic cancer.

As used herein the “pharmaceutically acceptable carriers” useful in conjunction with therapeutic compounds disclosed herein can be conventional. Remington's Pharmaceutical Sciences, by E W. Martin, Mack Publishing Co., Easton, Pa., 19th Edition (1995), describes compositions and formulations suitable for pharmaceutical delivery of therapeutic agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, the terms “pharmaceutical” and “therapeutic agent” refer to a chemical compound or a composition capable of inducing a desired therapeutic or prophylactic effect when properly administered to a subject or a cell.

The term “replication-competent” as used herein refers to a virus, such as a myxoma virus, that is capable of infecting and replicating within a particular host cell, such as a human blood cell (e.g., a hematologic cancer cell, or a peripheral blood mononuclear cell).

The term “immunomodulatory transgene” refers to a genetic sequence that can be introduced into a virus genome and encodes a product that can affect the function of the immune system, for example, that affects inflammation, innate or adaptive immune signaling, innate or adaptive immune cell activation (e.g., target cell killing, production of cytokines, chemokines, or other inflammatory mediators), innate or adaptive immune cell homing (e.g., chemotaxis, extravasation, and/or accumulation at a site), innate or adaptive immune cell proliferation, innate or adaptive immune cell differentiation, antibody production, an anti-cancer immune response, or a combination thereof. Examples of immunomodulatory transgenes include, but are not limited to, STING, anti-PDL1, FAST, TNFα, and IL-12.

### Myxoma Virus

Myxoma virus (MYXV) is potentially well suited as a therapeutic virus against blood cancers, like multiple myeloma (MM), because of its unique biology. MYXV is a member of the poxviridae family and the leporipoxvirus genus (Chan W M, Rahman M M, and McFadden G. Oncolytic myxoma virus: the path to clinic. Vaccine. 2013; 31(39):4252-8, Chan W M, and McFadden G. Oncolytic Poxviruses. Annu Rev Virol. 2014; 1(1):119-41).

MYXV is a novel oncolytic virus that can target a variety of human and murine cancers, for example, both primary cancers and established cell lines (Stanford M M, and McFadden G. Myxoma virus and oncolytic virotherapy: a new biologic weapon in the war against cancer. Expert Opin Biol Ther. 2007; 7(9):1415-11425; Wang G, Barrett J W, Stanford M, Werden S J, Johnston J B, Gao X, et al. Infection of human cancer cells with myxoma virus requires Akt activation via interaction with a viral ankyrin-repeat host range factor. Proc Natl Acad Sci USA. 2006; 103(12):4640-5; Bartee E, Chan W M, Moreb J S, Cogle C R, and McFadden G. Selective purging of human multiple myeloma cells from autologous stem cell transplantation grafts using oncolytic myxoma virus. Biol Blood Marrow Transplant. 2012; 18(10):1540-51; Chan W M, Rahman M M, and McFadden G. Oncolytic myxoma virus: the path to clinic. Vaccine. 2013; 31(39):4252-8; Kim M, Madlambayan G J, Rahman M M, Smallwood S E, Meacham A M, Hosaka K, et al. Myxoma virus targets primary human leukemic stem and progenitor cells while sparing normal hematopoitic stem and progenitor cells. Leukemia. 2009; 32:2313-7; Villa N Y, Wasserfall C H, Meacham A M, Wise E, Chan W, Wingard J R, et al. Myxoma virus suppresses proliferation of activated T lymphocytes yet permits oncolytic virus transfer to cancer cells. Blood. 2015; 125(24):3778-88).

In nature, MYXV is rabbit-specific and generally does not cause infection or disease in immunocompetent humans, mice, or any other domestic animals. However, because of the nature of cancer pathway mutations associated with carcinogenesis, cancer cells from both mice and humans can exhibit a compromised ability to resist infection by some viruses, including MYXV (for example, compromised innate immune pathways) (Chan W M, and McFadden G. Oncolytic Poxviruses. Annu Rev Virol. 2014; 1(1):119-41, Sypula J, 'Wang F, Ma Y, Bell J, and McFadden G. Myxoma virus tropism in human tumors. Gene Ther and Mol Biol. 2004; 8:103-14).

Provided herein, in some embodiments, are modified (e.g., engineered) myxoma viruses (MYXV). The MYXV may be any virus that belongs to the Leporipoxvirus species of poxviruses that is replication-competent. The MYXV may be a wild-type strain of MYXV or it may be a genetically modified strain of MYXV. In some instances, the MYXV is Lausanne strain. In some instances, the MYXV is a South American MYXV strain that circulates in Sylvilagus brasiliensis. In some instances, the MYXV is a Californian MYXV strain that circulates in Sylvilagus bachmani. In some instances, the MYXV is 6918, an attenuated Spanish field strain that comprises modifications in genes M009L, M036L, M135R, and M148R (GenBank Accession number EU552530 which is hereby incorporated by reference as provided by GenBank on Aug. 27, 2019). In some instances, the MYXV is 6918VP60-T2 (GenBank Accession Number EU552531 which is hereby incorporated by reference as provided by GenBank on Aug. 27, 2019). In some instances, the MYXV is SG33, a strain comprising a genomic deletion that affects genes M151R, M152R, M153R, M154L, M156R, M008.1R, M008R, M007R, M006R, M005R, M004.1R, M004R, M003.2R, M003.1R, M002R, and M001R, (Collection Nationale de Cultures de Microorganismes (CNCM) Accession No. 1-1594). In some instances, the MYXV is a strain termed the Standard laboratory Strain (SLS).

In some instances, the MYXV genome comprises at least 70%, at least 75%, at least 80%, at least 85%, at least 90%, at least 95%, at least 96%, at least 97%, at least 98%, or at least 99%, such as between 95% and 98%, 95% and 99%, including 90%, 91%, 92%, 93%, 94%, 95%, 96%, 97%, 98%, or 99% sequence identity to a nucleic acid sequence disclosed in Cameron, et al., “The complete DNA sequence of Myxoma Virus,” Virology 264: 298-318 (1999). In some cases, the MYXV comprises the sequence disclosed in Cameron, et al., “The complete DNA sequence of Myxoma Virus,” Virology 264: 298-318 (1999).

The large and genetically stable poxvirus genome allows for genetic manipulation, for example, generation of viruses with one or more deletions and/or introduction of one or more therapeutic (e.g., immunomodulatory) transgenes (Nayerossadat N, Maedeh T, and Ali P A. Viral and nonviral delivery systems for gene delivery. Adv Biomed Res. 2012; 1:27).

Provided herein, in some embodiments, are myxoma viruses (MYXV) and modified (e.g., engineered) MYXV. The MYXV may be any virus that belongs to the Leporipoxvirus species of pox viruses that is replication-competent. The MYXV may be a wild-type strain of MYXV or it may be a genetically modified strain of MYXV.

The Myxoma virus genome can be modified to express one or more therapeutic transgenes (e.g., immunomodulatory transgenes, such as STING, anti-PDL1, FAST, TNFα, and/or IL-12) using molecular biology techniques disclosed herein and/or known to a skilled person, and described for example in Sambrook et al. ((2001) Molecular Cloning: a Laboratory Manual, 3rd ed., Cold Spring Harbour Laboratory Press). A skilled person will be able to determine which portions of the Myxoma viral genome can be deleted such that the virus is still capable of productive infection, for example, to provide a replication competent virus. For example, non-essential regions of the viral genome that can be deleted can be deduced from comparing the published viral genome sequence with the genomes of other well-characterized viruses (see for example C. Cameron, S. Hota-Mitchell, L. Chen, J. Barrett, J.-X. Cao, C. Macaulay, D. Willer, D. Evans, and G. McFadden, Virology (1999) 264: 298-318)).

In some embodiments, the disclosed MYXV recombinant construct is an oncolytic viral candidate to treat relapsed/refractory primary human hematologic malignancies such as multiple myeloma (MM) and to target and reduce or eliminate minimal residual disease (MRD). In some embodiments, the MYXV comprises one or more transgenes.

In some embodiments, a MYXV of the disclosure comprises one or more gene modifications, deletions, and/or disruptions in the MYXV genome. For example, a MYXV of the disclosure can comprise one or more insertions, deletions, or substitutions within or adjacent to one or more genes in the genome. An insertion, deletion or modification can comprise a gene knockout (for example, deletion of one or more nucleotides that thereby reduces or eliminates functionality of the product encoded by the gene, or insertion of one or more nucleotides that thereby disrupt expression and/or function of the product encoded by the gene). In some embodiments, an insertion, deletion, or modification does not comprise a gene knockout (for example, a sequence can be inserted at an intergenic locus between two genes, without disrupting expression of the two genes). A modification can be, for example, a transgene replacing a portion of a gene disclosed herein.

In some embodiments, a MYXV of the disclosure comprises one or more insertions, deletions, or substitutions within or adjacent to one or more genes associated with the ability of the virus to cause disease in a host animal. In some embodiments, a MYXV of the disclosure comprises one or more insertions, deletions, or substitutions within or adjacent to one or more genes associated with host cell tropism. In some embodiments, a MYXV of the disclosure comprises one or more insertions, deletions, or substitutions within or adjacent to one or more genes associated with the ability of the virus to evade an innate immune response. In some embodiments, a MYXV of the disclosure comprises one or more insertions, deletions, or substitutions within or adjacent to one or more genes that modulate immune signaling in an infected cell (e.g., cytokine receptor signaling). In some embodiments, a MYXV of the disclosure comprises one or more insertions, deletions, or substitutions within or adjacent to one or more genes that modulate a cell death pathway in an infected cell (e.g., a gene that codes for a product that promotes or inhibits apoptosis, such as M011L). In some embodiments, a MYXV of the disclosure comprises one or more insertions, deletions, or substitutions within or adjacent to one or more genes that modulates viral replication in a cancer cell (e.g., increases or decreases the rate of viral replication in a cancer cell).

In some embodiments, the one or more genes associated with the ability of the virus to cause disease in a host animal, associated with host cell tropism, associated with the ability of the virus to evade an innate immune response, that can modulate immune signaling in an infected cell, that can modulate a cell death pathway in an infected cell, that can modulate viral replication in a cancer cell, or a combination thereof, comprise any one or more of M001R, M002R, M003.1R, M003.2R, M004.1R, M004R, M005R, M006R, M007R, M008.1R, M008R, M009L, M013, M036L, M063L, M011L, M128L, M131R, M135R, M136R, M141R, M148R, M151R, M152R, M153R, M154L, M156R, M-T2, M-T4, M-T5, M-T7, and SOD.

In some embodiments, a MYXV of the disclosure comprises a modification of a MYXV gene. In some instances, the modification is a deletion that impairs the function of a protein encoded by the MYXV gene. In some cases, the modification is a partial deletion. For example, a partial deletion can be an at least 10%, at least 20%, at least 30%, at least 40%, at least 50%, at least 60%, at least 70%, at least 80%, at least 90%, or at least 95% deletion of the MYXV gene. In some embodiments, a partial deletion can be an at most 10%, at most 20%, at most 30%, at most 40%, at most 50%, at most 60%, at most 70%, at most 80%, at most 90%, or at most 95% deletion of the MYXV gene. In other cases, the modification is a full deletion of the MYXV gene. In some embodiments, the modification is a replacement of the MYXV gene with one or more transgenes of the disclosure (e.g., STING, anti-PDL1, FAST, TNFα, and/or IL-12).

In some embodiments, a MYXV of the disclosure comprises one or more insertions, deletions, or substitutions within or adjacent to one or more genes associated with host cell tropism (for example, rabbit cell tropism). In some embodiments, one or more genes associated with rabbit cell tropism comprises M011L, M063, M135R, M136R, M-T2, M-T4, M-T5, M-T7, or a combination thereof. In some instances, the one or more genes associated with rabbit cell tropism comprise M135R, M136R, or a combination thereof.

In some embodiments, a MYXV of the disclosure comprises a modification of the M135R gene. In some embodiments, the MYXV comprises a partial deletion or full deletion of M135R gene. A deletion or disruption of the M135R gene can, for example, attenuate the ability of a MYXV of the disclosure to cause disease in a host animal, without impairing the ability of the MYXV to exhibit an anti-cancer effect (e.g., infect and kill cancer cells).

In some instances, the modification is a deletion that impairs the function of a protein encoded by the M135R gene. In some cases, the modification is a partial deletion (e.g., at least 10%, at least 20%, at least 30%, at least 40%, at least 50%, at least 60%, at least 70%, at least 80%, at least 90%, at least 95% deletion, at most 10%, at most 20%, at most 30%, at most 40%, at most 50%, at most 60%, at most 70%, at most 80%, at most 90%, at most 95%, about 10%, about 20%, about 30%, about 40%, about 50%, about 60%, about 70%, about 80%, about 90%, or about 95% deletion) of the M135R gene. In other cases, the modification is a full deletion of the M135R gene. In some embodiments, the deletion is a deletion of at least 1, at least 2, at least 3, at least 4, at least 5, at least 7, at least 10, at least 20, at least 30, at least 40, at least 50, at least 60, at least 70, at least 100, at least 200, or at least 300 nucleic acids. In some embodiments, the deletion disrupts a promoter (e.g., a promoter that drives expression of M135R in a wild type MYXV). In some embodiments, the deletion introduces a stop codon into the M135R gene sequence, for example, a premature stop codon that prevents expression of a full length M135R transcript and/or protein.

In some embodiments, the MYXV comprises a modification of M135R gene that impairs the function of M135R gene (e.g., insertion of a sequence that disrupts the expression and/or function of the M135R gene). In some embodiments, the insertion is an insertion of at least 1, at least 2, at least 3, at least 4, at least 5, at least 7, at least 10, at least 20, at least 30, at least 40, at least 50, at least 60, at least 70, at least 100, at least 200, at least 300, at least 400, at least 500, at least 600, at least 700, at least 800, at least 900, at least 1000, at least 1500, or at least 2000 nucleic acids. In some embodiments, the insertion alters the reading frame of the M135R gene sequence, thereby disrupting expression of the M135R transcript and/or protein.

In some instances, the mutation is a substitution, for example, a substitution that attenuates an activity or expression level of a protein encoded by the M135R gene. In some embodiments, at least 1, at least 2, at least 3, at least 4, at least 5, at least 7, at least 10, at least 20, at least 30 nucleic acids are substituted. In some embodiments, the substitution introduces a stop codon into the M135R gene sequence, for example, a premature stop codon that prevents expression of a full length M135R transcript and/or protein. In some embodiments, the substitution disrupts a promoter (e.g., a promoter that drives expression of M135R in a wild type MYXV).

In some embodiments, a modification or mutation disclosed herein attenuates the activity level of the M135R gene and/or protein by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a MYXV that encodes a functional wild type M135R.

In some embodiments, a modification or mutation disclosed herein attenuates the expression level of the M135R gene and/or protein by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a MYXV that encodes a functional wild type M135R.

In some embodiments, a MYXV disclosed herein has an activity level of the M135R protein that is attenuated by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a MYXV that encodes a functional wild type M135R.

In some embodiments, a MYXV disclosed herein has an expression level of the M135R gene and/or protein that is attenuated by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a MYXV that encodes a functional wild type M135R.

In some embodiments, a transgene of the disclosure replaces the M135R gene within the MYXV genome (for example, disrupts or replaces the M135R gene with one or more transgenes of the disclosure (e.g., STING, anti-PDL1, FAST, TNFα, and/or IL-12). In some embodiments, a transgene of the disclosure replaces a portion of the M135R gene within the MYXV genome (for example, replaces a portion of the M135R gene with STING, anti-PDL1, FAST, TNFα, and/or IL-12). In some embodiments, a transgene of the disclosure is inserted between M135R gene and M136R gene within the MYXV genome. In some embodiments, a transgene of the disclosure is inserted in the M135-136 locus. For MYXV, 136 as used herein can refer to M136 gene locus of the MYXV. In some embodiments, M136 refers to M136R of MYXV.

In some embodiments, a MYXV of the disclosure comprises a modification of the M153 gene. The M153 gene product is an E3-Ubiquitin ligase that may participate in the down regulation of diverse cellular receptors and proteins, for example, degradation of MHC Class I and CD4 in human cells. In some embodiments, a MYXV of the disclosure has an attenuated activity and/or expression level of M153 protein. In some embodiments, an attenuated activity and/or expression level of M153 protein can enhance presentation of immune epitopes, for example, MHC-dependent presentation of viral and/or cancer immune peptides. Enhanced presentation of immune epitopes by infected cancer cells can elicit stronger immune responses, including anti-cancer T cell responses, such as anti-cancer CD8+ T cell responses. In some embodiments, an attenuated activity and/or expression level of M153 protein increases direct antigen presentation from M153KO virus-infected tumor cells by MHC-I, and enhances immune activation mediated by the MYXV.

In some embodiments, the MYXV comprises a partial deletion or full deletion of M153 gene. In some instances, the modification is a deletion that impairs the function of a protein encoded by the M153 gene. In some cases, the modification is a partial deletion (e.g., at least 10%, at least 20%, at least 30%, at least 40%, at least 50%, at least 60%, at least 70%, at least 80%, at least 90%, at least 95% deletion, at most 10%, at most 20%, at most 30%, at most 40%, at most 50%, at most 60%, at most 70%, at most 80%, at most 90%, at most 95%, about 10%, about 20%, about 30%, about 40%, about 50%, about 60%, about 70%, about 80%, about 90%, or about 95% deletion) of the M153 gene. In some embodiments, the deletion is a deletion of at least 1, at least 2, at least 3, at least 4, at least 5, at least 7, at least 10, at least 20, at least 30, at least 40, at least 50, at least 60, at least 70, at least 100, at least 200, or at least 300 nucleic acids. In some embodiments, the deletion disrupts a promoter (e.g., a promoter that drives expression of M153 in a wild type MYXV). In some embodiments, the deletion introduces a stop codon into the M153 gene sequence, for example, a premature stop codon that prevents expression of a full length M153 transcript and/or protein.

In other cases, the modification is a full deletion of the M153 gene (e.g., deletion of entire coding region of M153 gene, deletion of entire M153 gene, etc.). In some embodiments, the MYXV comprises a modification of M153 gene that impairs the function of M153 gene (e.g., insertion of a sequence that disrupts the expression and/or function of the M153 gene). In some embodiments, the insertion is an insertion of at least 1, at least 2, at least 3, at least 4, at least 5, at least 7, at least 10, at least 20, at least 30, at least 40, at least 50, at least 60, at least 70, at least 100, at least 200, at least 300, at least 400, at least 500, at least 600, at least 700, at least 800, at least 900, at least 1000, at least 1500, or at least 2000 nucleic acids. In some embodiments, the insertion alters the reading frame of the M153 gene sequence, thereby disrupting expression of the M153 transcript and/or protein.

In some instances, the mutation is a substitution, for example, a substitution that attenuates an activity or expression level of a protein encoded by the M153 gene. In some embodiments, at least 1, at least 2, at least 3, at least 4, at least 5, at least 7, at least 10, at least 20, at least 30 nucleic acids are substituted. In some embodiments, the substitution introduces a stop codon into the M153 gene sequence, for example, a premature stop codon that prevents expression of a full length M153 transcript and/or protein. In some embodiments, the substitution disrupts a promoter (e.g., a promoter that drives expression of M153 in a wild type MYXV).

In some embodiments, a modification or mutation disclosed herein attenuates the activity level of the M153 gene and/or protein by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a MYXV that encodes a functional wild type M153.

In some embodiments, a modification or mutation disclosed herein attenuates the expression level of the M153 gene and/or protein by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a MYXV that encodes a functional wild type M153.

In some embodiments, a MYXV disclosed herein has an activity level of the M153 protein that is attenuated by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a MYXV that encodes a functional wild type M153.

In some embodiments, a MYXV disclosed herein has an expression level of the M153 gene and/or protein that is attenuated by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a MYXV that encodes a functional wild type M153.

In some embodiments, a transgene of the disclosure replaces the M153 gene within the MYXV genome (for example, disrupts or replaces the M153 gene with one or more transgenes of the disclosure (e.g., STING, anti-PDL1, FAST, TNFα, and/or IL-12). In some embodiments, a transgene of the disclosure replaces a portion of the M153 gene within the MYXV genome (for example, replaces a portion of the M153 gene with STING, anti-PDL1, FAST, TNFα, and/or IL-12).

### Transgenes

Provided herein, in some embodiments, are myxoma virus (MYXV) recombinant constructs comprising transgenes.

In the context of cancer and the tumor microenvironment, a range of immunomodulatory factors can affect the interplay between cancer cells and the immune system. For example, immunomodulatory factors can directly interact with malignant cells, positively or negatively affect infiltration of cytotoxic lymphocytes into the tumor microenvironment, positively or negatively affect the production of cytokines and chemokines that mediate immune responses against cancer cells, or a combination thereof. One or more immunomodulatory transgenes can be introduced into the MYXV genome, for example, to promote an immune response that more effectively treats or reduces a cancer.

For example, in some embodiments, one or more MYXV endogenous genes that regulate different forms of immune modulation and/or cell death are ablated. In some embodiments, one or more therapeutic immunomodulatory transgenes are introduced to the viral genome (e.g., to increase immunogenicity and/or induce preferred forms of cancer cell death). In some embodiments, one or more MYXV endogenous genes are ablated, and one or more immunomodulatory transgenes are introduced to the viral genome.

In some embodiments, the transgene is an immunomodulatory transgene. In some embodiments, a MYXV of the disclosure comprises STING, anti-PDL1, FAST, TNFα, IL-12, or a combination thereof.

Disclosed herein, in some embodiments, are recombinant MYXV constructs that express an immunomodulatory transgene that participates in an immune signaling pathway (e.g., an innate immune signaling pathway, such as a signaling pathway downstream of a pattern recognition receptor). In some embodiments, a MYXV of the disclosure encodes Stimulator of interferon genes (STING). Generation of anti-tumor cellular responses might benefit from the cloning and expression in MYXV vectors of transgenes such as STING, which is involved in pathways that elicit cellular responses. Activation of the STING pathway and subsequent secretion of type I interferons directly can correlate with elicitation of cellular immune responses. STING has been proposed as a crucial component of the innate immune sensing of tumors, and its activation is linked to anti-tumor cellular responses. Activation of the STING pathway can also promote anti-cancer adaptive immune responses.

The STING pathway can be activated in the presence of cytosolic DNA that is detected by the sensor cyclic GMP-AMP synthase (cGAS). Upon binding DNA, cGAS can generate cyclic GMP-amp (cGAMP) which binds and activates STING. Intratumor injection of STING agonist has shown strong therapeutic effects in several cancer mice models. Combining STING pathway activation with an oncolytic virus that selectively infects cancer cells, such as MYXV, may lead to improved anti-tumor activity. Additionally, in some cases STING can induce autophagy, which can contribute to cell death.

Table 1 provides examples of STING sequences. SEQ ID NO: 1 provides a DNA sequence and SEQ ID NO: 2 provides an amino acid sequence of STING. In some cases, STING can be modified to generate a constitutively active STING (e.g., one that does not require or rely on activation by cGAMP). In some embodiments, a STING of the disclosure is a constitutively active variant of STING. SEQ ID NO: 3 provides a DNA sequence and SEQ ID NO: 4 provides an amino acid sequence of a constitutively active STING.

TABLE 1

| SEQ | | |
|---|---|---|
| ID | | |
| NO: | Description | Sequence |
| 1 | STING | ATGCCCCACTCCAGCCTGCATCCATCCATC |
| | | CCGTGTCCCAGGGGTCACGGGGCCCAGAAG |
| | | GCAGCCTTGGTTCTGCTGAGTGCCTGCCTG |
| | | GTGACCCTTTGGGGGCTAGGAGAGCCACCA |
| | | GAGCACACTCTCCGGTACCTGGTGCTCCAC |
| | | CTAGCCTCCCTGCAGCTGGGACTGCTGTTA |
| | | AACGGGGTCTGCAGCCTGGCTGAGGAGCTG |
| | | CGCCACATCCACTCCAGGTACCGGGGCAGC |
| | | TACTGGAGGACTGTGCGGGCCTGCCTGGGC |
| | | TGCCCCCTCCGCCGTGGGGCCCTGTTGCTG |
| | | CTGTCCATCTATTTCTACTACTCCCTCCCA |
| | | AATGCGGTCGGCCCGCCCTTCACTTGGATG |
| | | CTTGCCCTCCTGGGCCTCTCGCAGGCACTG |
| | | AACATCCTCCTGGGCCTCAAGGGCCTGGCC |
| | | CCAGCTGAGATCTCTGCAGTGTGTGAAAAA |
| | | GGGAATTTCAACGTGGCCCATGGGCTGGCA |
| | | TGGTCATATTACATCGGATATCTGCGGCTG |
| | | ATCCTGCCAGAGCTCCAGGCCCGGATTCGA |
| | | ACTTACAATCAGCATTACAACAACCTGCTA |
| | | CGGGGTGCAGTGAGCCAGCGGCTGTATATT |
| | | CTCCTCCCATTGGACTGTGGGGTGCCTGAT |
| | | AACCTGAGTATGGCTGACCCCAACATTCGC |
| | | TTCCTGGATAAACTGCCCCAGCAGACCGGT |
| | | GACCATGCTGGCATCAAGGATCGGGTTTAC |
| | | AGCAACAGCATCTATGAGCTTCTGGAGAAC |
| | | GGGCAGCGGGCGGGCACCTGTGTCCTGGAG |
| | | TACGCCACCCCCTTGCAGACTTTGTTTGCC |
| | | ATGTCACAATACAGTCAAGCTGGCTTTAGC |
| | | CGGGAGGATAGGCTTGAGCAGGCCAAACTC |
| | | TTCTGCCGGACACTTGAGGACATCCTGGCA |
| | | GATGCCCCTGAGTCTCAGAACAACTGCCGC |
| | | CTCATTGCCTACCAGGAACCTGCAGATGAC |
| | | AGCAGCTTCTCGCTGTCCCAGGAGGTTCTC |
| | | CGGCACCTGCGGCAGGAGGAAAAGGAAGAG |
| | | GTTACTGTGGGCAGCTTGAAGACCTCAGCG |
| | | GTGCCCAGTACCTCCACGATGTCCCAAGAG |
| | | CCTGAGCTCCTCATCAGTGGAATGGAAAAG |
| | | CCCCTCCCTCTCCGCACGGATTTCTCTTGA |
| 2 | STING | MPHSSLHPSIPCPRGHGAQKAALVLLSACL |
| | | VTLWGLGEPPEHTLRYLVLHLASLQLGLLL |
| | | NGVCSLAEELRHIHSRYRGSYWRTVRACLG |
| | | CPLRRGALLLLSIYFYYSLPNAVGPPFTWM |
| | | LALLGLSQALNILLGLKGLAPAEISAVCEK |
| | | GNFNVAHGLAWSYYIGYLRLILPELQARIR |
| | | TYNQHYNNLLRGAVSQRLYILLPLDCGVPD |
| | | NLSMADPNIRFLDKLPQQTGDHAGIKDRVY |
| | | SNSIYELLENGQRAGTCVLEYATPLQTLFA |
| | | MSQYSQAGFSREDRLEQAKLFCRTLEDILA |
| | | DAPESQNNCRLIAYQEPADDSSFSLSQEVL |
| | | RHLRQEEKEEVTVGSLKTSAVPSTSTMSQE |
| | | PELLISGMEKPLPLRTDFS |
| 3 | Constitutively | ATGCCCCACTCCAGCCTGCATCCATCCATC |
| | active STING | CCGTGTCCCAGGGGTCACGGGGCCCAGAAG |
| | (852G/T) | GCAGCCTTGGTTCTGCTGAGTGCCTGCCTG |
| | | GTGACCCTTTGGGGGCTAGGAGAGCCACCA |
| | | GAGCACACTCTCCGGTACCTGGTGCTCCAC |
| | | CTAGCCTCCCTGCAGCTGGGACTGCTGTTA |
| | | AACGGGGTCTGCAGCCTGGCTGAGGAGCTG |
| | | CGCCACATCCACTCCAGGTACCGGGGCAGC |
| | | TACTGGAGGACTGTGCGGGCCTGCCTGGGC |
| | | TGCCCCCTCCGCCGTGGGGCCCTGTTGCTG |
| | | CTGTCCATCTATTTCTACTACTCCCTCCCA |
| | | AATGCGGTCGGCCCGCCCTTCACTTGGATG |
| | | CTTGCCCTCCTGGGCCTCTCGCAGGCACTG |
| | | AACATCCTCCTGGGCCTCAAGGGCCTGGCC |
| | | CCAGCTGAGATCTCTGCAGTGTGTGAAAAA |
| | | GGGAATTTCAACGTGGCCCATGGGCTGGCA |
| | | TGGTCATATTACATCGGATATCTGCGGCTG |
| | | ATCCTGCCAGAGCTCCAGGCCCGGATTCGA |
| | | ACTTACAATCAGCATTACAACAACCTGCTA |
| | | CGGGGTGCAGTGAGCCAGCGGCTGTATATT |
| | | CTCCTCCCATTGGACTGTGGGGTGCCTGAT |
| | | AACCTGAGTATGGCTGACCCCAACATTCGC |
| | | TTCCTGGATAAACTGCCCCAGCAGACCGGT |
| | | GACCATGCTGGCATCAAGGATCGGGTTTAC |
| | | AGCAACAGCATCTATGAGCTTCTGGAGAAC |
| | | GGGCAGCGGGCGGGCACCTGTGTCCTGGAG |
| | | TACGCCACCCCCTTGCAGACTTTGTTTGCC |
| | | ATGTCACAATACAGTCAAGCTGGCTTTAGC |
| | | CGGGAGGATAGTCTTGAGCAGGCCAAACTC |
| | | TTCTGCCGGACACTTGAGGACATCCTGGCA |
| | | GATGCCCCTGAGTCTCAGAACAACTGCCGC |
| | | CTCATTGCCTACCAGGAACCTGCAGATGAC |
| | | AGCAGCTTCTCGCTGTCCCAGGAGGTTCTC |
| | | CGGCACCTGCGGCAGGAGGAAAAGGAAGAG |
| | | GTTACTGTGGGCAGCTTGAAGACCTCAGCG |
| | | GTGCCCAGTACCTCCACGATGTCCCAAGAG |
| | | CCTGAGCTCCTCATCAGTGGAATGGAAAAG |
| | | CCCCTCCCTCTCCGCACGGATTTCTCTTGA |
| 4 | Constitutively | MPHSSLHPSIPCPRGHGAQKAALVLLSACL |
| | active STING | VTLWGLGEPPEHTLRYLVLHLASLQLGLLL |
| | (R284S) | NGVCSLAEELRHIHSRYRGSYWRTVRACLG |
| | | CPLRRGALLLLSIYFYYSLPNAVGPPFTWM |
| | | LALLGLSQALNILLGLKGLAPAEISAVCEK |
| | | GNFNVAHGLAWSYYIGYLRLILPELQARIR |
| | | TYNQHYNNLLRGAVSQRLYILLPLDCGVPD |
| | | NLSMADPNIRFLDKLPQQTGDHAGIKDRVY |
| | | SNSIYELLENGQRAGTCVLEYATPLQTLFA |
| | | MSQYSQAGFSREDSLEQAKLFCRTLEDILA |
| | | DAPESQNNCRLIAYQEPADDSSFSLSQEVL |
| | | RHLRQEEKEEVTVGSLKTSAVPSTSTMSQE |
| | | PELLISGMEKPLPLRTDFS |

In some embodiments, a MYXV of the disclosure encodes a STING that comprises, consists essentially of, or consists of an amino acid sequence with at least about 70%, at least about 80%, at least about 90%, at least about 95%, at least about 97%, or at least about 99% sequence identity to SEQ ID NO: 2 or 4. In some embodiments, a MYXV of the disclosure encodes a STING that comprises, consists essentially of, or consists of an amino acid sequence that is SEQ ID NO: 2 or SEQ ID NO: 4. In some embodiments, a MYXV of the disclosure comprises a nucleotide sequence that comprises, consists essentially of, or consists of a sequence with at least about 70%, at least about 80%, at least about 90%, at least about 95%, at least about 97%, or at least about 99% sequence identity to SEQ ID NO: 1 or 3. In some embodiments, a MYXV of the disclosure comprises a nucleotide sequence that comprises, consists essentially of, or consists of a sequence that is SEQ ID NO: 1 or 3. A MYXV of the disclosure can encode a human STING.

Disclosed herein, in some embodiments, are recombinant MYXV constructs that express an immunomodulatory transgene that promotes syncytia formation, such as a fusion-associated small membrane protein (FAST). In some cases, FAST enhances death of MYXV-infected cells.

In some cases, a MYXV of the disclosure comprises or encodes a p14 FAST. p14 FAST protein is a small (14-kDa, 375-bp) fusogenic protein from a reptilian reovirus. In some cases, p14 FAST can enhance lateral virus spread between host cells through its ability to promote cell-cell fusion. A non-limiting example of a p14 FAST amino acid sequence is provide in Table 2.

TABLE 2

| SEQ | | |
|---|---|---|
| ID | | |
| NO: | Description | Sequence |
| 5 | P14 FAST | MGSGPSNFVNHAPGEAIVTGLE |
| | | KGADKVAGTISHTIWEVIAGLV |
| | | ALLTFLAFGFWLFKYLQKRRER |
| | | RRQLTEFQKRYLRNSYRLSEIQ |
| | | RPISQHEYEDPYEPPSRRKPPP |
| | | PPYSTYVNIDNVSAI |

In some cases, a MYXV of the disclosure comprises (e.g., encodes) a FAST with the sequence of SEQ ID NO: 5. In some embodiments, a MYXV of the disclosure encodes a FAST that comprises, consists essentially of, or consists of an amino acid sequence with at least about 70%, at least about 80%, at least about 90%, at least about 95%, at least about 97%, or at least about 99% sequence identity to SEQ ID NO: 5. In some embodiments, a MYXV of the disclosure encodes a FAST that comprises, consists essentially of, or consists of an amino acid sequence that is SEQ ID NO: 5. A FAST of the disclosure can be a human FAST.

Disclosed herein, in some embodiments, are recombinant MYXV constructs that express an immunomodulatory transgene that encodes an antibody or antigen-binding fragment thereof that binds to PD-L1. In some cases, the antibody is antagonistic for PD-L1, and prevents interaction between PD-1 and PD-L1, allowing immune activation (e.g., activation of an anti-tumor immune response).

Non-limiting examples of amino acid sequences from an anti-PDL1 antibody, antigen-binding fragment thereof, or portions thereof are provided in Table 3.

TABLE 3

| SEQ | | |
|---|---|---|
| ID | | |
| NO: | Description | Sequence |
| 6 | Anti-PD-L1 | EVQLVESGGGLVQPGGSLRLSCAAS |
| | VH | GFTFSDSWIHWVRQAPGKGLEWVAW |
| | | ISPYGGSTYYADSVKGRFTISADTS |
| | | KNTAYLQMNSLRAEDTAVYYCARRH |
| | | WPGGFDYWGQGTLVTVSS |
| 7 | Anti-PD-L1 | DIQMTQSPSSLSASVGDRVTITCRA |
| | VL | SQDVSTAVAWYQQKPGKAPKLLIYS |
| | | ASFLYSGVPSRFSGSGSGTDFTLTI |
| | | SSLQPEDFATYYCQQYLYHPATFGQ |
| | | GTKVEIK |
| 8 | Anti-PD-L1 | DSWIH |
| | HCDR1 | |
| 9 | Anti-PD-L1 | WISPYGGSTYYADSVKG |
| | HCDR2 | |
| 10 | Anti-PD-L1 | RHWPGGFDY |
| | HCDR3 | |
| 11 | Anti-PD-L1 | RASQDVSTAVA |
| | LCDR1 | |
| 12 | Anti-PD-L1 | SASFLYS |
| | LCDR2 | |
| 13 | Anti-PD-L1 | QQYLYHPAT |
| | LCDR3 | |

In some embodiments, a MYXV of the disclosure encodes an anti-PD-L1 that comprises, consists essentially of, or consists of an amino acid sequence with at least about 70%, at least about 80%, at least about 90%, at least about 95%, at least about 97%, or at least about 99% sequence identity to any one or more of SEQ ID NOs: 6-13. In some embodiments, a MYXV of the disclosure encodes an anti-PD-L1 that comprises, consists essentially of, or consists of an amino acid sequence that is any one or more of SEQ ID NOs: 6-13. In some cases, a MYXV of the disclosure comprises or encodes an anti-PD-L1 sequence that is of any one or more of SEQ ID NOs: 6-13 (e.g. a scFv comprising the CDRs and/or the variable domains in TABLE 3).

A sequence of the antibody- or antigen-binding fragment, including the CDRs and/or the variable domains thereof, can have at least 70% homology, at least 71% homology, at least 72% homology, at least 73% homology, at least 74% homology, at least 75% homology, at least 76% homology, at least 77% homology, at least 78% homology, at least 79% homology, at least 80% homology, at least 81% homology, at least 82% homology, at least 83% homology, at least 84% homology, at least 85% homology, at least 86% homology, at least 87% homology, at least 88% homology, at least 89% homology, at least 90% homology, at least 91% homology, at least 92% homology, at least 93% homology, at least 94% homology, at least 95% homology, at least 96% homology, at least 97% homology, at least 98% homology, at least 99% homology, at least 99.1% homology, at least 99.2% homology, at least 99.3% homology, at least 99.4% homology, at least 99.5% homology, at least 99.6% homology, at least 99.7% homology, at least 99.8% homology, at least 99.9% homology, at least 99.91% homology, at least 99.92% homology, at least 99.93% homology, at least 99.94% homology, at least 99.95% homology, at least 99.96% homology, at least 99.97% homology, at least 99.98% homology, or at least 99.99% homology to an amino acid or nucleic acid sequence disclosed herein.

Disclosed herein, in some embodiments, are recombinant MYXV constructs that express an immunomodulatory transgene that encodes a cytokine that can upregulate an immune response (e.g., an anti-cancer immune response). In some cases, a MYXV construct of the disclosure comprises a sequence that codes for TNF alpha (TNF). In some cases, a MYXV construct of the disclosure comprises a sequence that codes for IL-12.

Non-limiting examples of TNFα and IL-12 sequences are provided in Table 4.

TABLE 4

| SEQ | | |
|---|---|---|
| ID | | |
| NO: | Description | Sequence |
| 14 | TNFα, | MSTESMIRDVELAEEALPKK |
| | membrane | TGGPQGSRRCLFLSLFSFLI |
| | form | VAGATTLFCLLHFGVIGPQR |
| | | EEFPRDLSLISPLAQAVRSS |
| | | SRTPSDKPVAHVVANPQAEG |
| | | QLQWLNRRANALLANGVELR |
| | | DNQLVVPSEGLYLIYSQVLF |
| | | KGQGCPSTHVLLTHTISRIA |
| | | VSYQTKVNLLSAIKSPCQRE |
| | | TPEGAEAKPWYEPIYLGGVF |
| | | QLEKGDRLSAEINRPDYLDF |
| | | AESGQVYFGIIAL |
| 15 | TNFα, | VRSSSRTPSDKPVAHVVANP |
| | soluble | QAEGQLQWLNRRANALLANG |
| | form | VELRDNQLVVPSEGLYLIYS |
| | | QVLFKGQGCPSTHVLLTHTI |
| | | SRIAVSYQTKVNLLSAIKSP |
| | | CQRETPEGAEAKPWYEPIYL |
| | | GGVFQLEKGDRLSAEINRPD |
| | | YLDFAESGQVYFGIIAL |
| 16 | IL-12A | MCPARSLLLVATLVLLDHLS |
| | | LARNLPVATPDPGMFPCLHH |
| | | SQNLLRAVSNMLQKARQTLE |
| | | FYPCTSEEIDHEDITKDKTS |
| | | TVEACLPLELTKNESCLNSR |
| | | ETSFITNGSCLASRKTSFMM |
| | | ALCLSSIYEDLKMYQVEFKT |
| | | MNAKLLMDPKRQIFLDQNML |
| | | AVIDELMQALNFNSETVPQK |
| | | SSLEEPDFYKTKIKLCILLH |
| | | AFRIRAVTIDRVMSYLNAS |
| 17 | IL-12A | RNLPVATPDPGMFPCLHHSQ |
| | lacking | NLLRAVSNMLQKARQTLEFY |
| | signal | PCTSEEIDHEDITKDKTSTV |
| | peptide | EACLPLELTKNESCLNSRET |
| | | SFITNGSCLASRKTSFMMAL |
| | | CLSSIYEDLKMYQVEFKTMN |
| | | AKLLMDPKRQIFLDQNMLAV |
| | | IDELMQALNFNSETVPQKSS |
| | | LEEPDFYKTKIKLCILLHAF |
| | | RIRAVTIDRVMSYLNAS |
| 18 | IL-12B | MCHQQLVISWFSLVFLASPL |
| | | VAIWELKKDVYVVELDWYPD |
| | | APGEMVVLTCDTPEEDGITW |
| | | TLDQSSEVLGSGKTLTIQVK |
| | | EFGDAGQYTCHKGGEVLSHS |
| | | LLLLHKKEDGIWSTDILKDQ |
| | | KEPKNKTFLRCEAKNYSGRF |
| | | TCWWLTTISTDLTFSVKSSR |
| | | GSSDPQGVTCGAATLSAERV |
| | | RGDNKEYEYSVECQEDSACP |
| | | AAEESLPIEVMVDAVHKLKY |
| | | ENYTSSFFIRDIIKPDPPKN |
| | | LQLKPLKNSRQVEVSWEYPD |
| | | TWSTPHSYFSLTFCVQVQGK |
| | | SKREKKDRVFTDKTSATVIC |
| | | RKNASISVRAQDRYYSSSWS |
| | | EWASVPCS |
| 19 | IL-12B | IWELKKDVYVVELDWYPDAP |
| | lacking | GEMVVLTCDTPEEDGITWTL |
| | signal | DQSSEVLGSGKTLTIQVKEF |
| | peptide | GDAGQYTCHKGGEVLSHSLL |
| | | LLHKKEDGIWSTDILKDQKE |
| | | PKNKTFLRCEAKNYSGRFTC |
| | | WWLTTISTDLTFSVKSSRGS |
| | | SDPQGVTCGAATLSAERVRG |
| | | DNKEYEYSVECQEDSACPAA |
| | | EESLPIEVMVDAVHKLKYEN |
| | | YTSSFFIRDIIKPDPPKNLQ |
| | | LKPLKNSRQVEVSWEYPDTW |
| | | STPHSYFSLTFCVQVQGKSK |
| | | REKKDRVFTDKTSATVICRK |
| | | NASISVRAQDRYYSSSWSEW |
| | | ASVPCS |

In some embodiments, a MYXV of the disclosure encodes a cytokine that comprises, consists essentially of, or consists of an amino acid sequence with at least about 70%, at least about 80%, at least about 90%, at least about 95%, at least about 97%, or at least about 99% sequence identity to any one or more of SEQ ID NOs: 14-19. In some embodiments, a MYXV of the disclosure encodes a cytokine that comprises, consists essentially of, or consists of an amino acid sequence that is any one or more of SEQ ID NOs: 14-19. In some cases, a MYXV of the disclosure comprises or encodes a cytokine sequence that is any one or more of SEQ ID NOs: 14-19.

Disclosed herein, in some embodiments, are recombinant MYXV constructs that are armed with one or more of these immunomodulatory transgenes to target blood cancers, including MM. In this disclosure, MYXV expressing immunomodulatory transgenes are shown to selectively infect and kill primary human MM cells from patients with refractory disease that are resistant to standard therapies. In addition, it is demonstrated that these virus constructs can compromise MM cell viability by inducing apoptosis and death of MM cells. Notably, two kinds of MM cell killing can be observed: direct cytotoxic killing of virus-infected MM cells, plus “off-target” killing of un-infected MM cells. Without wishing to be bound by theory, killing of uninfected MM cells may be mediated by MYXV-activated immune cells, for example, immune cells that are activated by the product of the immunomodulatory transgene.

A sequence of the disclosure can have at least 70% homology, at least 71% homology, at least 72% homology, at least 73% homology, at least 74% homology, at least 75% homology, at least 76% homology, at least 77% homology, at least 78% homology, at least 79% homology, at least 80% homology, at least 81% homology, at least 82% homology, at least 83% homology, at least 84% homology, at least 85% homology, at least 86% homology, at least 87% homology, at least 88% homology, at least 89% homology, at least 90% homology, at least 91% homology, at least 92% homology, at least 93% homology, at least 94% homology, at least 95% homology, at least 96% homology, at least 97% homology, at least 98% homology, at least 99% homology, at least 99.1% homology, at least 99.2% homology, at least 99.3% homology, at least 99.4% homology, at least 99.5% homology, at least 99.6% homology, at least 99.7% homology, at least 99.8% homology, at least 99.9% homology, at least 99.91% homology, at least 99.92% homology, at least 99.93% homology, at least 99.94% homology, at least 99.95% homology, at least 99.96% homology, at least 99.97% homology, at least 99.98% homology, or at least 99.99% homology to an amino acid or nucleic acid sequence disclosed herein.

A transgene (e.g., an anti-PDL1) of the disclosure can encode an antigen-binding protein, for example, one or more variable regions or complementarity determining regions (CDRs) from an antibody. In some embodiments, a transgene (e.g., an anti-PDL1) of the disclosure comprises one or more single chain variable fragments (scFvs) derived from one or more antibodies. A scFv (single-chain variable fragment) is a fusion protein that can comprise the VH and VL domains of an antibody connected by a peptide linker. For example, a transgene can comprise two scFvs to allow binding of two targets.

Antigen binding proteins can be engineered based on antibody variable regions or CDRs. The variable (V) regions of an antibody mediate antigen binding and define the specificity of a particular antibody for an antigen. The variable region comprises relatively invariant sequences called framework regions, and hypervariable regions, which differ considerably in sequence among antibodies of different binding specificities. Within hypervariable regions are amino acid residues that primarily determine the binding specificity of the antibody. Sequences comprising these residues are known as complementarity determining regions (CDRs). One antigen binding site of an antibody comprises six CDRs, three in the hypervariable regions of the light chain, and three in the hypervariable regions of the heavy chain. The CDRs in the light chain are designated L1, L2, and L3, while the CDRs in the heavy chain are designated H1, H2, and H3. CDRs can also be designated LCDR1, LCDR2, LCDR3, HCDR1, HCDR2, and HCDR3, respectively. The contribution of each CDR to antigen binding varies among antibodies. CDRs can vary in length. For example, CDRs are often 5 to 14 residues in length, but CDRs as short as 0 residues or as long as 25 residues or longer exist. Several methods can be used to predict or designate CDR sequences, for example, the Kabat, Chothia, IMGT, paratome, Martin, and AHo methods. These CDR prediction methods can use different numbering systems, for example, because sequence insertions and deletions are numbered differently.

An antigen-binding protein or fragment can comprise a portion of an antibody, for example, the antigen-binding or variable region of the intact antibody. Non-limiting examples of antibody fragments include Fab, Fab′, F(ab′)2, dimers and trimers of Fab conjugates, Fv, scFv, minibodies, dia-, tria-, and tetrabodies, and linear antibodies. Fab and Fab′ are antigen-binding fragments that can comprise the VH and CH1 domains of the heavy chain linked to the VL and CL domains of the light chain via a disulfide bond. A F(ab′)2 can comprise two Fab or Fab′ that are joined by disulfide bonds. A Fv can comprise the VH and VL domains held together by non-covalent interactions. A scFv (single-chain variable fragment) is a fusion protein that can comprise the VH and VL domains connected by a peptide linker. Manipulation of the orientation of the VH and VL domains and the linker length can be used to create different forms of molecules that can be monomeric, dimeric (diabody), trimeric (triabody), or tetrameric (tetrabody).

In some embodiments, a transgene of the disclosure can encode a linker sequence (e.g., a linker sequence between different domains of a protein encoded by the transgene). In some embodiments, a linker is used to join antibody variable regions to form an scFv. In some embodiments, a linker is used to join two scFvs to form a multi-specific construct. A linker sequence can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues in length. In some embodiments, a linker is at least 1, at least 3, at least 5, at least 7, at least 9, at least 11, or at least 15 amino acids in length. In some embodiments, a linker is at most 5, at most 7, at most 9, at most 11, at most 15, at most 20, at most 25, or at most 50 amino acids in length.

A flexible linker can have a sequence containing stretches of glycine and serine residues. The small size of the glycine and serine residues provides flexibility, and allows for mobility of the connected functional domains. The incorporation of serine or threonine can maintain the stability of the linker in aqueous solutions by forming hydrogen bonds with the water molecules, thereby reducing unfavorable interactions between the linker and protein moieties. Flexible linkers can also contain additional amino acids such as threonine and alanine to maintain flexibility, as well as polar amino acids such as lysine and glutamine to improve solubility. A rigid linker can have, for example, an alpha helix-structure. An alpha-helical rigid linker can act as a spacer between protein domains. A linker can comprise any of the sequences in Table 5, or repeats thereof (e.g., 2, 3, 4, 5, 6, 7, 8, 9, or 10 repeats of any of SEQ ID NOs: 20-29). SEQ ID NOs: 20-25 provide flexible linkers. SEQ ID NOs: 26-29 provide rigid linkers.

TABLE 5

| SEQ ID NO: | Sequence |
|---|---|
| 20 | GGGGS |
| 21 | GGGS |
| 22 | GG |
| 23 | KESGSVSSEQLAQFRSLD |
| 24 | EGKSSGSGSESKST |
| 25 | GSAGSAAGSGEF |
| 26 | EAAAK |
| 27 | EAAAR |
| 28 | PAPAP |
| 29 | AEAAAKEAAAKA |

In some embodiments, a MYXV of the disclosure can comprise one or more additional transgenes (e.g., one or more transgenes in addition to one or more of STING, anti-PDL1, FAST, TNFα, and/or IL-12).

In some embodiments a MYXV of the disclosure can increase autophagy in cells that are infected by the MYXV. Autophagy can be increased, for example, by an engineered MYXV that expresses an immunomodulatory transgene, relative to a MYXV that lacks the transgene, or relative to uninfected cells. In some embodiments, autophagy is increased by at least 5%, at least 10%, at least 20%, at least 30%, at least 40%, at least 50%, at least 75%, at least 2-fold, at least three-fold, at least five-fold, or at least ten-fold, as determined by an LC3-I to LC3-II conversion assay.

In some embodiments a MYXV of the disclosure can increase killing of infected cancer cells (e.g., “on-target” killing). Killing of infected cancer cells can be increased, for example, by an engineered MYXV that expresses an immunomodulatory transgene, relative to a MYXV that lacks the transgene, or relative to uninfected cancer cells. A MYXV of the disclosure can increase killing of infected cancer cells, for example, by at least 5%, at least 10%, at least 20%, at least 30%, at least 40%, at least 50%, at least 75%, at least 2-fold, at least three-fold, at least five-fold, or at least ten-fold, e.g., as determined by a live/dead staining assay. The MYXV can preferentially infect and preferentially kill cancer cells over non-cancer cells.

In some embodiments a MYXV of the disclosure can increase killing of uninfected cancer cells (e.g., “off-target” killing). Killing of uninfected cancer cells can be increased, for example, by an engineered MYXV that expresses an immunomodulatory transgene, relative to a MYXV that lacks the transgene, or relative to uninfected cancer cells. A MYXV of the disclosure can increase killing of uninfected cancer cells, for example, by at least 5%, at least 10%, at least 20%, at least 30%, at least 40%, at least 50%, at least 75%, at least 2-fold, at least three-fold, at least five-fold, or at least ten-fold, e.g., as determined by a live/dead staining assay. The MYXV can preferentially infect and preferentially kill cancer cells over non-cancer cells.

In some embodiments, a MYXV of the disclosure comprises one immunomodulatory transgene. In some embodiments, a MYXV of the disclosure comprises two immunomodulatory transgenes. In some embodiments, a MYXV of the disclosure comprises three immunomodulatory transgenes. In some embodiments, a MYXV of the disclosure comprises four immunomodulatory transgenes. In some embodiments, a MYXV of the disclosure comprises five immunomodulatory transgenes.

In some embodiments, a MYXV of the disclosure can comprise one or more non-immunomodulatory transgenes (e.g., one or more non-immunomodulatory transgenes in addition to one or more of STING, anti-PDL1, FAST, TNFα, and/or IL-12).

In some embodiments, a MYXV of the disclosure can comprise one or more reporter transgenes (e.g., one or more reporter transgenes in addition to one or more of STING, anti-PDL1, FAST, TNFα, and/or IL-12). A reporter transgene (or reporter gene) can be used to monitor or quantify a MYXV in vitro, ex vivo, or in vivo. In some embodiments, a reporter transgene can be used to identify cells infected by an MYXV of the disclosure. For example, a MYXV of the disclosure can express a fluorescent transgene, and infected cells can be identified via fluorescence (e.g., fluorescence microscopy or flow cytometry). In some embodiments, a reporter transgene can be used to quantify cells infected by an MYXV of the disclosure. For example, a MYXV of the disclosure can express a fluorescent transgene, and infected cells can be quantified via fluorescence (e.g., quantification of the number or proportion of infected cells via fluorescence microscopy or flow cytometry). In some embodiments, a reporter transgene can be used to quantify viral replication or viral load in cells infected by an MYXV of the disclosure. For example, a MYXV of the disclosure can express a fluorescent transgene, and infected cells can be quantified via fluorescence (e.g., quantification of the average fluorescence intensity of cells via flow cytometry of fluorescence microscopy). In some embodiments, a MYXV of the disclosure can express a reporter gene that can be used for quantifying viral load or viral replication in vivo (e.g., imaging using an in vivo imaging system (IVIS)).

A reporter transgene of the disclosure can be expressed constitutively (e.g., under control of a constitutive promoter). A reporter transgene of the disclosure can be expressed conditionally (e.g., expressed under the control of a conditional promoter, e.g., a promoter that is only active or is more active in certain phases of a replication cycle).

Non-limiting examples of reporter transgenes include fluorescent proteins (e.g., green fluorescent protein (GFP), TdTomato, cyan fluorescent protein (CFP), yellow fluorescent protein (YFP), red fluorescent protein (RFP), Verde fluorescent protein (VFP), kindling fluorescent protein (KFP), mCherry, mTangerine, mRaspberry, mPlum, DsRed, etc.) and enzymes and substrates involved in luminescence (e.g., luciferin and/or luciferase).

In some embodiments, a MYXV of the disclosure does not comprise or encode a reporter transgene (e.g., does not encode any fluorescent or luminescent proteins).

In addition to expression of one or more immunomodulatory transgenes, a MYXV of the disclosure can be modified to carry one or more other genes that can enhance the anticancer effect of the MYXV treatment. Such a gene may be a gene that is involved in triggering apoptosis, or is involved in targeting the infected cell for immune destruction, such as a gene that restores responsiveness of the cell to interferon, or that results in the expression of a cell surface marker that stimulates an antibody response, such as a bacterial cell surface antigen. A MYXV of the disclosure can be modified to express one or more genes involved in shutting off the neoplastic or cancer cell's proliferation and growth, thereby preventing or reducing division of the cancer cell. In some embodiments, a MYXV of the disclosure can be modified to include therapeutic genes, such as genes involved in the synthesis of chemotherapeutic agents. In some embodiments, a MYXV of the disclosure can comprise a transgene that increases viral replication in cells of a particular species (for example, increased replication in human cancer cells for increased killing and inhibition of human cancer cells).

### Methods of Treatment

Provided herein, in some embodiments, are methods of treating a hematological cancer in a subject utilizing a myxoma virus (MYXV) of the disclosure. The hematological cancer can be a hematological cancer that comprises minimal residual disease (MRD) and/or drug-resistant MRD.

As disclosed in the Examples below, in vitro studies have demonstrated the ability of MYXV constructs of the disclosure to significantly eliminate refractory primary human multiple myeloma (MM) cells from patients who have failed standard therapies. Studies performed with MYXV have shown it can be a highly specific anti-cancer agent with a tropism for a number of human and murine cancer types.

Treatments of the disclosure can comprise a number of novel and advantageous aspects. For example, these virus constructs are so far the only described oncolytic viruses that selectively target and directly eliminate drug-resistant primary human MM cells that have been directly infected by each virus (e.g., CD138+ cells that express a viral reporter gene, such as GFP+ or TdTomato+). In some embodiments, MYXV of the disclosure comprising one or more immunomodulatory transgenes can not only eliminate hematologic cancer cells by direct killing of virus-infected cells, but also can eliminate disease by enhanced “off-target” killing of uninfected cancer cells (e.g., via virus-activated immune cells). In some embodiments, MYXV of the disclosure comprising transgenes can elicit increased killing of uninfected cancer cells compared to other viruses (e.g., unarmed viruses or viruses lacking the immunomodulatory transgene(s)). In some embodiments, MYXV of the disclosure can exhibit enhanced “off-target” killing of uninfected MM cells (e.g., CD138+ cells that are negative for a viral reporter gene, such as GFP− or TdTomato−). Without wishing to be bound by any specific theory, virus-enhanced killing of uninfected cells may be mediated by MYXV-activated immune cells (e.g., immune cells activated by a transgene of the disclosure, other viral components, or a combination thereof).

The use of MYXV to treat hematologic malignancies (e.g., refractory and/or minimal residual disease (MRD) of hematologic malignancies) can comprise multiple advantages over current therapies including chemotherapy and stem cell transplantation, and over other candidate oncolytic viruses. MYXV comprises a limited tropism that can, for example, allow the virus to infect human cancer cells, but not allow the virus to infect non-cancerous human cells. Unlike most viruses adapted from human pathogens, MYXV does not cause disease in humans, making it safe even for those patients with compromised immune systems. The lack of pre-existing anti-MYXV adaptive immunity in the human population can be advantageous, for example, allowing the virus to infect and kill cancer cells without being cleared as rapidly as a virus adapted from a human pathogen.

In ex vivo treatment approaches disclosed herein, incubation of MYXV with cells (e.g., bone marrow (BM) cells and/or peripheral blood mononuclear cells (PBMCs)) can be fast, for example, requiring only 1 hour of virus incubation ex vivo before re-infusion of the cells back into the cancer patient.

Thus, aspects of the present disclosure provide a method for inhibiting and/or treating a hematological cancer in a subject in need thereof. In certain embodiments, the method includes administering to a subject, such as a human subject, a MYXV of the disclosure that expresses one or more immunomodulatory transgenes, thereby treating and/or inhibiting the hematological cancer in the subject in need thereof. The subject can be a mammal. The subject can be a human.

In some embodiments, the MYXV comprises MYXV-STING. In some embodiments, the MYXV comprises MYXV-anti-PD-L1. In some embodiments, the MYXV comprises MYXV-FAST. In some embodiments, the MYXV comprises MYXV-TNFα. In some embodiments, the MYXV comprises MYXV-IL-12.

In some embodiments, an MYXV of the disclosure comprises a reporter transgene (e.g., a fluorescent protein or a luminescent substrate or enzyme). In some embodiments, an MYXV of the disclosure comprises one or more immunomodulatory transgenes, and further comprises a reporter transgene.

In some embodiments, a MYXV of the disclosure comprises a modification, insertion, deletion, or disruption in one or more genes in the viral genome. For example, a MYXV of the disclosure can comprise a modification, insertion, deletion, or disruption in or adjacent to any one or more of the M001R, M002R, M003.1R, M003.2R, M004.1R, M004R, M005R, M006R, M007R, M008.1R, M008R, M009L, M013, M036L, M063L, M011L, M128L, M131R, M135R, M136R, M141R, M148R, M151R, M152R, M153R, M154L, M156R, M-T2, M-T4, M-T5, M-T7, and SOD genes. In some embodiments, a deletion or disruption of a viral gene in a MYXV of the disclosure can reduce the ability of the virus to cause disease in a host animal, modulate host cell tropism, reduce innate immune evasion in non-cancer cells, modulate immune signaling in infected cells, modulate a cell death pathway in infected cells, increase viral replication in a cancer cells, or a combination thereof.

In some embodiments, a MYXV of the disclosure comprises one or more insertions, deletions, or substitutions within or adjacent to one or more genes associated with host cell tropism (for example, rabbit cell tropism). In some embodiments, one or more genes associated with rabbit cell tropism comprises M011L, M063, M135R, M136R, M-T2, M-T4, M-T5, M-T7, or a combination thereof. In some instances, the one or more genes associated with rabbit cell tropism comprise M135R, M136R, or a combination thereof.

In some embodiments, a MYXV of the disclosure comprises a modification, insertion, deletion, or disruption in the M135R gene. In some embodiments, a MYXV of the disclosure comprises a deletion or disruption in the M135R gene. A deletion or disruption of the M135R gene can, for example, attenuate the ability of a MYXV of the disclosure to cause disease in a host animal, without impairing the ability of the MYXV to exhibit an anti-cancer effect (e.g., infect and kill cancer cells, elicit an anti-tumor immune response, or a combination thereof). In some embodiments, a MYXV of the disclosure comprises a modification, insertion, deletion, or disruption in the M153 gene.

In some embodiments, a MYXV of the disclosure comprises a modification, insertion, deletion, or disruption in the SOD gene. In some embodiments, a MYXV of the disclosure comprises a deletion or disruption in the SOD gene.

MYXV can infect cells (e.g., human cells) that have a deficient innate anti-viral response. Having “a deficient innate anti-viral response” as used herein can refer to a cell that, when exposed to a virus or when invaded by a virus, fails to induce one or more anti-viral defense mechanisms. For example, a deficient innate anti-viral response can comprise failure to inhibit viral replication, failure to produce an anti-viral cytokine (e.g., an interferon), failure to respond to an anti-viral cytokine (e.g., induce an interferon response pathway), failure to induce apoptosis, failure to trigger recognition via an innate immune receptor (e.g., pattern recognition receptor), or a combination thereof.

A deficient innate anti-viral response may be caused by various factors, for example, malignant transformation, mutation, infection, genetic defect, or environmental stress.

In some embodiments, a MYXV of the disclosure is not administered to a subject comprising a deficient innate anti-viral response caused by a genetic defect, environmental stress, or an infection (e.g., a pre-existing infection with a different pathogen).

In some embodiments, a MYXV of the disclosure is administered to a subject comprising a deficient innate anti-viral response caused by malignant transformation (e.g., a cancer). A cell comprising a deficient innate anti-viral response can be a cancer cell, e.g., a cancer cell that has a reduced or defective innate anti-viral response upon exposure to or infection by a virus as compared to a normal cell, for example, a non-cancer cell. This can include, for example, a cancer cell that is non-responsive to interferon (e.g., type I interferon), and/or a cancer cell that has a reduced or defective apoptotic response or induction of the apoptotic pathway. In some embodiments of the method, an MYXV of the disclosure is capable of infecting a cell that has a deficient innate anti-viral response. In some embodiments, the cell is a mammalian cancer cell. In some embodiments, the cell is a human cancer cell, e.g., a human hematological cancer cell.

In some embodiments, a MYXV of the disclosure is used to treat a cancer. The examples provided herein for multiple myeloma are, by extension, applicable to other hematological cancers. Types of cancer that may be treated according to the disclosed method include, but are not limited to, hematological cancers such as leukemia, lymphoma, and myeloma, for example: multiple myeloma (MM); active multiple myeloma; smoldering multiple myeloma; plasmacytoma; solitary plasmacytoma of the bone; extramedullary plasmacytoma; light chain myeloma; non-secretory myeloma; immunoglobulin G (IgG) myeloma; immunoglobulin A (IgA) myeloma; immunoglobulin M (IgM) myeloma; immunoglobulin D (IgD) myeloma; immunoglobulin E (IgE) myeloma; hyperdiploid multiple myeloma; non-hyperdiploid multiple myeloma; Hodgkin lymphoma; non-Hodgkin lymphoma; acute lymphoblastic leukemia; acute myeloid leukemia; essential thrombocythemia; polycythemia vera; primary myelofibrosis; systemic mastocytosis; chronic myeloid leukemia; chronic neutrophilic leukemia; chronic eosinophilic leukemia; refractory anemia with ringed sideroblasts; refractory cytopenia with multilineage dysplasia; refractory anemia with excess blasts; type 1; refractory anemia with excess blasts; type 2; myelodysplastic syndrome (MDS) with isolated del (5q); MDS unclassifiable; chronic myelomonocytic leukemia (CML); atypical chronic myeloid leukemia; juvenile myelomonocytic leukemia; myeloproliferative/myelodysplastic syndromes-unclassifiable; B lymphoblastic leukemia/lymphoma; T lymphoblastic leukemia/lymphoma; diffuse large B-cell lymphoma; primary central nervous system lymphoma; primary mediastinal B-cell lymphoma; Burkitt lymphoma/leukemia; follicular lymphoma; chronic lymphocytic leukemia (CLL)/small lymphocytic lymphoma; B-cell prolymphocytic leukemia; lymphoplasmacytic lymphoma/Waldenstrom macroglobulinemia; Mantle cell lymphoma; marginal zone lymphomas; post-transplant lymphoproliferative disorders; HIV-associated lymphomas; primary effusion lymphoma; intravascular large B-cell lymphoma; primary cutaneous B-cell lymphoma; hairy cell leukemia; and monoclonal gammopathy of unknown significance; Anaplastic large cell lymphoma, Angioimmunoblastic T-cell lymphoma, Hepatosplenic T-cell lymphoma, B-cell lymphoma, reticuloendotheliosis, reticulosis, Mucosa-associated lymphatic tissue lymphoma, B-cell chronic lymphocytic leukemia, Waldenstrom's macroglobulinemia, Lymphomatoid granulomatosis, Nodular lymphocyte predominant Hodgkin's lymphoma, plasma cell leukemia, Acute erythraemia and erythroleukaemia, Acute erythremic myelosis, Acute erythroid leukemia, Heilmeyer-Schoner disease, Acute megakaryoblastic leukemia, Mast cell leukemia, Panmyelosis, Acute panmyelosis with myelofibrosis, Lymphosarcoma cell leukemia, Stem cell leukemia, Chronic leukaemia of unspecified cell type, Subacute leukaemia of unspecified cell type, Accelerated phase chronic myelogenous leukemia, Acute promyelocytic leukemia, Acute basophilic leukemia, Acute eosinophilic leukemia, Acute monocytic leukemia, Acute myeloblastic leukemia with maturation, Acute myeloid dendritic cell leukemia, Adult T-cell leukemia/lymphoma, Aggressive NK-cell leukemia, B-cell chronic lymphocytic leukemia, B-cell leukemia, Chronic myelogenous leukemia, Chronic idiopathic myelofibrosis, Kahler's disease, Myelomatosis, Solitary myeloma, Plasma cell leukemia, Angiocentric immunoproliferative lesion, Lymphoid granulomatosis, Angioimmunoblastic lymphadenopathy, T-gamma lymphoproliferative disease, Waldenstrom's macroglobulinaemia, Alpha heavy chain disease, Gamma heavy chain disease, and Franklin's disease. In some embodiments, the hematological cancer is multiple myeloma. In some embodiments, the cancer is a hematological cancer. In certain embodiments, the cancer comprises multiple myeloma.

Provided herein, in some embodiments, are methods of treating a hematological cancer (e.g., inhibiting, alleviating, stabilizing, reducing, or delaying progression of a hematological cancer). In some embodiments, the methods comprise administering a MYXV of the disclosure to a subject in need thereof to treat the hematological cancer. In some embodiments, the method further includes selecting a subject, such as a human subject, that has or is suspected of having a hematological cancer.

A MYXV of the disclosure can be administered in an amount effective to treat the hematological cancer. The amount may be sufficient to reduce the number of cancer cells in the subject (e.g., the concentration of the cancer cells in the subject's blood).

The effective amount to be administered to a subject can vary depending on many factors such as the pharmacodynamic properties of the MYXV, the modes of administration, the age, health and weight of the subject, the nature and extent of the disease state, the frequency of the treatment and the type of concurrent treatment, if any, and the virulence and titer of the virus.

The MYXV may be administered initially in a suitable amount that may be adjusted as required, depending on the clinical response of the subject. The effective amount of virus can be determined empirically and depends on the maximal amount of the MYXV that can be administered safely, and the minimal amount of the virus that produces the desired result.

To produce the same clinical effect when administering the virus systemically as that achieved through injection of the virus at the disease site, administration of significantly higher amounts of virus may be required. However, the appropriate dose level should be the minimum amount that would achieve the desired result.

The concentration of virus to be administered will vary depending on the virulence of the particular strain of MYXV that is to be administered and on the nature of the cells that are being targeted. In one embodiment, a dose of less than about 3×10\hat{ }10 focus forming units (“ffu”) or plaque-forming units (“pfu”), also called “infectious units”, is administered to a human subject, in various embodiments, between about 10\hat{ }2 to about 10\hat{ }9 ffu, between about 10\hat{ }2 to about 10\hat{ }7 ffu, between about 10\hat{ }3 to about 10\hat{ }6 ffu, or between about 10\hat{ }4 to about 10\hat{ }5 ffu may be administered in a single dose.

In some embodiments, a subject is administered a certain dose of focus forming units (FFU) or plaque forming units (PFU) of a MYXV of the disclosure.

In some embodiments, the dose of MYXV administered to a subject is at least 1×10\hat{ }2, 2×10\hat{ }2, 3×10\hat{ }2, 4×10\hat{ }2, 5×10\hat{ }2, 6×10\hat{ }2, 7×10\hat{ }2, 8×10\hat{ }2, 9×10\hat{ }2, 1×10\hat{ }3, 2×10\hat{ }3, 3×10\hat{ }3, 4×10\hat{ }3, 5×10\hat{ }3, 6×10\hat{ }3, 7×10\hat{ }3, 8×10\hat{ }3, 9×10\hat{ }3, 1×10\hat{ }4, 2×10\hat{ }4, 3×10\hat{ }4, 4×10\hat{ }4, 5×10\hat{ }4, 6×10\hat{ }4, 7×10\hat{ }4, 8×10\hat{ }4, 9×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, 2×10\hat{ }16, 3×10\hat{ }16, 4×10\hat{ }16, 5×10\hat{ }16, 6×10\hat{ }16, 7×10\hat{ }16, 8×10\hat{ }16, 9×10\hat{ }16, 1×10\hat{ }17, 2×10\hat{ }17, 3×10\hat{ }17, 4×10\hat{ }17, 5×10\hat{ }17, 6×10\hat{ }17, 7×10\hat{ }17, 8×10\hat{ }17, 9×10\hat{ }17, 1×10\hat{ }18, 2×10\hat{ }18, 3×10\hat{ }18, 4×10\hat{ }18, 5×10\hat{ }18, 6×10\hat{ }18, 7×10\hat{ }18, 8×10\hat{ }18, 9×10\hat{ }18, 1×10\hat{ }19, 2×10\hat{ }19, 3×10\hat{ }19, 4×10\hat{ }19, 5×10\hat{ }19, 6×10\hat{ }19, 7×10\hat{ }19, 8×10\hat{ }19, 9×10\hat{ }19, 1×10\hat{ }20, 2×10\hat{ }20, 3×10\hat{ }20, 4×10\hat{ }20, 5×10\hat{ }20, 6×10\hat{ }20, 7×10\hat{ }20, 8×10\hat{ }20, or 9×10\hat{ }20 FFU or PFU of a MYXV of the disclosure.

In some embodiments, the dose of MYXV administered to a subject is at most 1×10\hat{ }2, 2×10\hat{ }2, 3×10\hat{ }2, 4×10\hat{ }2, 5×10\hat{ }2, 6×10\hat{ }2, 7×10\hat{ }2, 8×10\hat{ }2, 9×10\hat{ }2, 1×10\hat{ }3, 2×10\hat{ }3, 3×10\hat{ }3, 4×10\hat{ }3, 5×10\hat{ }3, 6×10\hat{ }3, 7×10\hat{ }3, 8×10\hat{ }3, 9×10\hat{ }3, 1×10\hat{ }4, 2×10\hat{ }4, 3×10\hat{ }4, 4×10\hat{ }4, 5×10\hat{ }4, 6×10\hat{ }4, 7×10\hat{ }4, 8×10\hat{ }4, 9×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }01, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, 2×10\hat{ }16, 3×10\hat{ }16, 4×10\hat{ }16, 5×10\hat{ }16, 6×10\hat{ }16, 7×10\hat{ }16, 8×10\hat{ }16, 9×10\hat{ }16, 1×10\hat{ }17, 2×10\hat{ }17, 3×10\hat{ }17, 4×10\hat{ }17, 5×10\hat{ }17, 6×10\hat{ }17, 7×10\hat{ }17, 8×10\hat{ }17, 9×10\hat{ }17, 1×10\hat{ }18, 2×10\hat{ }18, 3×10\hat{ }18, 4×10\hat{ }18, 5×10\hat{ }18, 6×10\hat{ }18, 7×10\hat{ }18, 8×10\hat{ }18, 9×10\hat{ }18, 1×10\hat{ }19, 2×10\hat{ }19, 3×10\hat{ }19, 4×10\hat{ }19, 5×10\hat{ }19, 6×10\hat{ }19, 7×10\hat{ }19, 8×10\hat{ }19, 9×10\hat{ }19, 1×10\hat{ }20, 2×10\hat{ }20, 3×10\hat{ }20, 4×10\hat{ }20, 5×10\hat{ }20, 6×10\hat{ }20, 7×10\hat{ }20, 8×10\hat{ }20, or 9×10\hat{ }20 FFU or PFU of a MYXV of the disclosure.

In some embodiments, a subject is administered a certain dose of focus forming units (FFU) or plaque forming units (PFU) of a MYXV of the disclosure per kilogram of body weight.

In some embodiments, the dose of MYXV administered to a subject is at least 1×10\hat{ }2, 2×10\hat{ }2, 3×10\hat{ }2, 4×10\hat{ }2, 5×10\hat{ }2, 6×10\hat{ }2, 7×10\hat{ }2, 8×10\hat{ }2, 9×10\hat{ }2, 1×10\hat{ }3, 2×10\hat{ }3, 3×10\hat{ }3, 4×10\hat{ }3, 5×10\hat{ }3, 6×10\hat{ }3, 7×10\hat{ }3, 8×10\hat{ }3, 9×10\hat{ }3, 1×10\hat{ }4, 2×10\hat{ }4, 3×10\hat{ }4, 4×10\hat{ }4, 5×10\hat{ }4, 6×10\hat{ }4, 7×10\hat{ }4, 8×10\hat{ }4, 9×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, 2×10\hat{ }16, 3×10\hat{ }16, 4×10\hat{ }16, 5×10\hat{ }16, 6×10\hat{ }16, 7×10\hat{ }16, 8×10\hat{ }16, 9×10\hat{ }16, 1×10\hat{ }17, 2×10\hat{ }17, 3×10\hat{ }17, 4×10\hat{ }17, 5×10\hat{ }17, 6×10\hat{ }17, 7×10\hat{ }17, 8×10\hat{ }17, 9×10\hat{ }17, 1×10\hat{ }18, 2×10\hat{ }18, 3×10\hat{ }18, 4×10\hat{ }18, 5×10\hat{ }18, 6×10\hat{ }18, 7×10\hat{ }18, 8×10\hat{ }18, 9×10\hat{ }18, 1×10\hat{ }19, 2×10\hat{ }19, 3×10\hat{ }19, 4×10\hat{ }19, 5×10\hat{ }19, 6×10\hat{ }19, 7×10\hat{ }19, 8×10\hat{ }19, 9×10\hat{ }19, 1×10\hat{ }20, 2, 2×10\hat{ }20, 3×10\hat{ }20, 4×10\hat{ }20, 5×10\hat{ }20, 6×10\hat{ }20, 7×10\hat{ }20, 8×10\hat{ }20, or 9×10\hat{ }20 FFU or PFU of a MYXV of the disclosure per kilogram of body weight of the subject.

In some embodiments, the dose of MYXV administered to a subject is at most 1×10\hat{ }2, 2×10\hat{ }2, 3×10\hat{ }2, 4×10\hat{ }2, 5×10\hat{ }2, 6×10\hat{ }2, 7×10\hat{ }2, 8×10\hat{ }2, 9×10\hat{ }2, 1×10\hat{ }3, 2×10\hat{ }3, 3×10\hat{ }3, 4×10\hat{ }3, 5×10\hat{ }3, 6×10\hat{ }3, 7×10\hat{ }3, 8×10\hat{ }3, 9×10\hat{ }3, 1×10\hat{ }4, 2×10\hat{ }4, 3×10\hat{ }4, 4×10\hat{ }4, 5×10\hat{ }4, 6×10\hat{ }4, 7×10\hat{ }4, 8×10\hat{ }4, 9×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, 2×10\hat{ }16, 3×10\hat{ }16, 4×10\hat{ }16, 5×10\hat{ }16, 6×10\hat{ }16, 7×10\hat{ }16, 8×10\hat{ }16, 9×10\hat{ }16, 1×10\hat{ }17, 2×10\hat{ }17, 3×10\hat{ }17, 4×10\hat{ }17, 5×10\hat{ }17, 6×10\hat{ }17, 7×10\hat{ }17, 8×10\hat{ }17, 9×10\hat{ }17, 1×10\hat{ }18, 2×10\hat{ }18, 3×10\hat{ }18, 4×10\hat{ }18, 5×10\hat{ }18, 6×10\hat{ }18, 7×10\hat{ }18, 8×10\hat{ }18, 9×10\hat{ }18, 1×10\hat{ }19, 2×10\hat{ }19, 3×10\hat{ }19, 4×10\hat{ }19, 5×10\hat{ }19, 6×10\hat{ }19, 7×10\hat{ }19, 8×10\hat{ }19, 9×10\hat{ }19, 1×10\hat{ }20, 2×10\hat{ }20, 3×10\hat{ }20, 4×10\hat{ }20, 5×10\hat{ }20, 6×10\hat{ }20, 7×10\hat{ }20, 8×10\hat{ }20, or 9×10\hat{ }20 FFU or PFU of a MYXV of the disclosure per kilogram of body weight of the subject.

A MYXV of the disclosure can be administered at any interval desired. In some embodiments, the MYXV can be administered hourly. In some embodiments, the MYXV can be administered about every 1, 2, 3, 4, 5, 6, 7, 8, 9, 10, 11, 12, 13, 14, 15, 16, 18, 20, 22, 24, 26, 28, 30, 32, 36, 40, 44, or 48 hours. In some embodiments, the MYXV can be administered twice a day, once a day, five times a week, four times a week, three times a week, two times a week, once a week, once every two weeks, once every three weeks, once every four weeks, once a month, once every five weeks, once every six weeks, once every eight weeks, once every two months, once every twelve weeks, once every three months, once every four months, once every six months, once a year, or less frequently.

A MYXV of the disclosure can be administered to a subject in a therapeutically-effective amount by various forms and routes including, for example, systemic, oral, topical, parenteral, intravenous injection, intravenous infusion, intratumoral injection, subcutaneous injection, intramuscular injection, intradermal injection, intraperitoneal injection, intracerebral injection, subarachnoid injection, intraspinal injection, intrasternal injection, intraarticular injection, endothelial administration, local administration, intranasal administration, intrapulmonary administration, intraarterial administration, intrathecal administration, inhalation, intralesional administration, intradermal administration, epidural administration, absorption through epithelial or mucocutaneous linings (e.g., oral mucosa, rectal and intestinal mucosa), intracapsular administration, subcapsular administration, intracardiac administration, transtracheal administration, subcuticular administration, subarachnoid administration, subcapsular administration, intraspinal administration, or intrasternal administration.

In some embodiments, the virus is administered systemically. In some embodiments, the virus is administered by injection at a disease site. In some embodiments, the virus is administered orally. In some embodiments, the virus is administered parenterally.

A MYXV of the disclosure (e.g., expressing one or more immunomodulatory transgenes) can be administered as a sole therapy or can be administered in combination with one or more other therapies. In some embodiments, a MYXV of the disclosure is administered in combination with a chemotherapy, an immunotherapy, a cell therapy, a radiation therapy, a stem cell transplant (such as an autologous stem cell transplant), or a combination thereof. For example, the MYXV expressing one or more immunomodulatory transgenes may be administered either prior to or following another treatment, such as administration of radiotherapy or conventional chemotherapeutic drugs and/or a stem cell transplant, such as an autologous stem cell transplant or an allogenic stem cell transplant (e.g., a HLA-matched, HLA-mismatched, or haploidentical transplant).

In some embodiments, a MYXV of the disclosure can be in combination with an immune checkpoint modulator. Examples of immune checkpoint modulators include, but are not limited to, PD-L1 inhibitors such as durvalumab (Imfinzi) from AstraZeneca, atezolizumab (MPDL3280A) from Genentech, avelumab from EMD Serono/Pfizer, CX-072 from CytomX Therapeutics, FAZ053 from Novartis Pharmaceuticals, KN035 from 3D Medicine/Alphamab, LY3300054 from Eli Lilly, or M7824 (anti-PD-L1/TGFbeta trap) from EMD Serono; PD-L2 inhibitors such as GlaxoSmithKline's AMP-224 (Amplimmune), and rHIgM12B7; PD-I inhibitors such as nivolumab (Opdivo) from Bristol-Myers Squibb, pembrolizumab (Keytruda) from Merck, AGEN 2034 from Agenus, BGB-A317 from BeiGene, B1-754091 from Boehringer-Ingelheim Pharmaceuticals, CBT-501 (genolimzumab) from CBT Pharmaceuticals, INCSHR1210 from Incyte, JNJ-63723283 from Janssen Research & Development, MEDIO680 from MedImmune, MGA 012 from MacroGenics, PDR001 from Novartis Pharmaceuticals, PF-06801591 from Pfizer, REGN2810 (SAR439684) from Regeneron Pharmaceuticals/Sanofi, or TSR-042 from TESARO; CTLA-4 inhibitors such as ipilimumab (also known as Yervoy®, MDX-010, BMS-734016 and MDX-101) from Bristol Meyers Squibb, tremelimumab (CP-675,206, ticilimumab) from Pfizer, or AGEN 1884 from Agenus; LAG3 inhibitors such as BMS-986016 from Bristol-Myers Squibb, IMP701 from Novartis Pharmaceuticals, LAG525 from Novartis Pharmaceuticals, or REGN3767 from Regeneron Pharmaceuticals; B7-H3 inhibitors such as enoblituzumab (MGA271) from MacroGenics; KIR inhibitors such as Lirilumab (IPH2101; BMS-986015) from Innate Pharma; CD137 inhibitors such as urelumab (BMS-663513, Bristol-Myers Squibb), PF-05082566 (anti-4-1BB, PF-2566, Pfizer), or XmAb-5592 (Xencor); and PS inhibitors such as Bavituximab. In some embodiments, the MYXV is combined with an antibody or antigen-binding fragment thereof, an RNAi molecule, or a small molecule, that acts on or is specific for, for example, TIM3, CD52, CD30, CD20, CD33, CD27, OX40, GITR, ICOS, BTLA (CD272), CD160, 2B4, LAIR1, TIGHT, LIGHT, DR3, CD226, CD2, or SLAM.

An MYXV of the disclosure can be prepared using standard techniques. For example, the virus may be prepared by infecting cultured rabbit cells, or immortalized permissive human or primate cells, with the MYXV strain that is to be used, allowing the infection to progress such that the virus replicates in the cultured cells, and can be released by standard methods for disrupting the cell surface and thereby releasing the virus particles for harvesting. Once harvested, the virus titer may be determined, for example, by infecting a confluent lawn of rabbit cells and performing a plaque assay (see Mossman et al. (1996) Virology 215:17-30 which is hereby incorporated by reference in its entirety).

### Cellular Delivery of MYXV

Further disclosed herein, in some embodiments, is a novel delivery strategy where a MYXV of the disclosure is first adsorbed to cells, and the cells are administered to a subject. This method can deliver a MYXV of the disclosure to sites of disease via virus-bearing “carrier” cells. In some embodiments, this cell-assisted delivery of virus has the ability to reduce or eliminate tumor burden and increase survival of the subject.

The delivery of MYXV via carrier cells represents a new potential therapeutic regimen for hematological cancers. In some embodiments, a MYXV of the disclosure is adsorbed to a plurality of cells (for example, leukocytes from bone marrow and/or peripheral blood), and the leukocytes are infused into a subject. Pre-loading leukocytes with MYXV ex vivo prior to leukocyte infusion into a cancer-bearing recipient can be exploited for multiple myeloma (MM) and for any other hematologic cancers disclosed herein. In some embodiments, pre-loading leukocytes with MYXV ex vivo prior to leukocyte infusion into a cancer-bearing recipient can be effective for treating any cancer amenable to the localization and infiltration of the leukocytes into distant tumor sites.

In some embodiments, the combined “leukocyte/MYXV” therapy causes increased cancer cell death in the tumor beds to enhance anti-tumor immunogenicity. For example, in some embodiments a MYXV of the disclosure (e.g., a MYXV expressing one or more immunomodulatory transgenes) is delivered to cancer sites such as the bone marrow beds that harbor minimal residual disease (MRD), via migration of leukocytes pre-adsorbed or pre-infected with virus ex vivo. This systemic delivery method is sometimes called “ex vivo virotherapy”, or EVV (e.g., EV2), because the virus is first delivered to leukocytes prior to infusion into the patient.

In some embodiments, the cell-mediated delivery of MYXV increases the level of direct killing of infected hematological cancer cells, and, while not being bound by theory, acts as an activator of the host immune system, which can lead to long term regression of cancer. This can provide a new method of treatment of hematological cancers in the bone and/or lymph nodes, which has proved to be difficult with current treatments.

Thus, in certain embodiments, methods of the disclosure comprise administering to a subject with cancer leukocytes that comprise an adsorbed MYXV of the disclosure (e.g., a MYXV that comprises one or more immunomodulatory transgenes), thereby treating and/or inhibiting the cancer in the subject. A MYXV of the disclosure can be adsorbed by exposing a plurality of cells to the MYXV under conditions that permit binding of the MYXV to the surface of the plurality of cells.

In some embodiments, a MYXV of the disclosure is adsorbed to leukocytes (for example, leukocytes from bone marrow and/or peripheral blood), and the leukocytes are infused into a subject. The leukocytes can be from bone marrow (for example, from bone marrow aspirate or bone marrow biopsy). The leukocytes can be from blood (e.g., peripheral blood mononuclear cells). In some embodiments, the leukocytes are obtained from a subject, for example a subject that has cancer, adsorbed with MYXV, and re-infused into the subject (e.g., as an autologous cell transplant). In some embodiments, the leukocytes are obtained from a subject's tissue. In some embodiments, the leukocytes are obtained from one or more allogenic donors (for example, HLA-matched, HLA-mismatched, or haploidentical donors). In some embodiments, the leukocytes are obtained from an HLA-matched sibling.

The leukocytes can be sorted or purified by, for example, red blood cell lysis, density gradient centrifugation (e.g., Ficoll-Paque), leukapheresis, techniques comprising antibodies or derivatives thereof (e.g., positive or negative selection via fluorescent activated cell sorting or magnetic activated cell sorting), or any combination thereof, before or after a MYXV of the disclosure is adsorbed. In some embodiments, the leukocytes are sorted or purified to enrich for cancer cells before or after a MYXV of the disclosure is adsorbed (e.g., cells expressing a marker associated with a cancer, e.g., CD138 for multiple myeloma cells). In some embodiments, the leukocytes are sorted or purified to enrich for non-cancer cells before or after a MYXV of the disclosure is adsorbed. In some embodiments, the cells are sorted or purified to enrich for one or more cell subsets before or after a MYXV of the disclosure is adsorbed (e.g., monocytes, lymphocytes, B cells, plasma cells, T cells, neutrophils, basophils, eosinophils, megakaryocytes, NK cells, NKT cells, mast cells, innate lymphoid cells, common myeloid precursors, common lymphoid precursors, myeloblasts, monoblasts, promonocytes, lymphoblasts, prolymphocytes, hemocytoblasts, megakaryoblasts, promegakaryocytes, stem cells, pro B cells, pre B cells, precursors thereof, or any combination thereof). In some embodiments, a MYXV of the disclosure is adsorbed to the leukocytes, and the leukocytes are enriched for cells comprising the MYXV (e.g., with MYXV bound and/or internalized).

The leukocytes can be stored (for example, cryopreserved) prior to or after adsorbing an MYXV of the disclosure. In some embodiments, the leukocytes can be cryopreserved, and later thawed prior to infusion into a subject.

In some embodiments, the method comprises adsorbing a MYXV of the disclosure onto the surface of leukocytes (e.g., peripheral blood mononuclear cells, bone marrow cells, or a purified/enriched subset thereof). In some embodiments, adsorbing the myxoma virus onto the surface of the leukocytes comprises exposing the leukocytes to the MYXV under conditions that permit binding of the MYXV to the surface of the mononuclear peripheral blood cells and/or bone marrow cells. In some embodiments, the method includes infecting the leukocytes with a MYXV of the disclosure. In some embodiments, infecting the leukocytes with a MYXV of the disclosure comprises exposing the leukocytes to the MYXV under conditions that permit internalization of the MYXV into at least a portion of the leukocytes. Exposing leukocytes to MYXV can comprise any suitable reagents or conditions (e.g., sterile cell culture media, media supplements, and appropriate incubation conditions to allow adsorption and/or infection of the leukocytes, and to maintain viability of the leukocytes).

The MYXV and leukocytes can be exposed to each other at any ratio that permits the virus to adsorb to the leukocytes. In some embodiments, adsorbing the myxoma virus onto the surface of the leukocytes comprises exposing the leukocytes to the MYXV at a multiplicity of infection (MOI) of about 0.000001, 0.00001, 0.0001, 0.0001, 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 400, 500, 600, 700, 800, 900, 1000, 2000, 3000, 4000, 5000, 6000, 7000, 8000, 9000, 1×10\hat{ }4, 1×10\hat{ }5, 1×10\hat{ }6, 1×10\hat{ }9, 1×10\hat{ }10, 1×10\hat{ }11, 1×10\hat{ }12, 1×10\hat{ }13, 1×10\hat{ }14, or 1×10\hat{ }15 viruses per leukocyte.

In some embodiments, adsorbing the myxoma virus onto the surface of the leukocytes comprises exposing the leukocytes to the MYXV at a multiplicity of infection (MOI) of at least 0.000001, at least 0.00001, at least 0.0001, at least 0.0001, at least 0.001, at least 0.01, at least 0.02, at least 0.03, at least 0.04, at least 0.05, at least 0.06, at least 0.07, at least 0.08, at least 0.09, at least 0.1, at least 0.2, at least 0.3, at least 0.4, at least 0.5, at least 0.6, at least 0.7, at least 0.8, at least 0.9, at least 1, at least 1.1, at least 1.2, at least 1.3, at least 1.4, at least 1.5, at least 1.6, at least 1.7, at least 1.8, at least 1.9, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, at least 150, at least 200, at least 250, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, at least 1×10\hat{ }4, at least 1×10\hat{ }5, at least 1×10\hat{ }6, at least 1×10\hat{ }9, at least 1×10\hat{ }10, at least 1×10\hat{ }11, at least 1×10\hat{ }12, at least 1×10\hat{ }13, at least 1×10\hat{ }14, or at least 1×10\hat{ }15 viruses per leukocyte.

In some embodiments, adsorbing the myxoma virus onto the surface of the leukocytes comprises exposing the leukocytes to the MYXV at a multiplicity of infection (MOI) of at most 0.000001, at most 0.00001, at most 0.0001, at most 0.0001, at most 0.001, at most 0.01, at most 0.02, at most 0.03, at most 0.04, at most 0.05, at most 0.06, at most 0.07, at most 0.08, at most 0.09, at most 0.1, at most 0.2, at most 0.3, at most 0.4, at most 0.5, at most 0.6, at most 0.7, at most 0.8, at most 0.9, at most 1, at most 1.1, at most 1.2, at most 1.3, at most 1.4, at most 1.5, at most 1.6, at most 1.7, at most 1.8, at most 1.9, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, at most 50, at most 60, at most 70, at most 80, at most 90, at most 100, at most 150, at most 200, at most 250, at most 300, at most 400, at most 500, at most 600, at most 700, at most 800, at most 900, at most 1000, at most 2000, at most 3000, at most 4000, at most 5000, at most 6000, at most 7000, at most 8000, at most 9000, at most 1×10\hat{ }4, at most 1×10\hat{ }5, at most 1×10\hat{ }6, at most 1×10\hat{ }9, at most 1×10\hat{ }10, at most 1×10\hat{ }11, at most 1×10\hat{ }12, at most 1×10\hat{ }13, at most 1×10\hat{ }14, or at most 1×10\hat{ }15 viruses per leukocyte.

In some embodiments, adsorbing the myxoma virus onto the surface of the leukocytes comprises exposing the leukocytes to the MYXV at a multiplicity of infection (MOI) of between about, for example, 0.000001 to 1×10\hat{ }15, 0.0001 to 1×10\hat{ }6, 0.001 to 1×10\hat{ }4, 0.001 to 1000, 0.001 to 100, 0.001 to 10, 0.001 to 1, 0.001 to 0.1, 0.001 to 0.01, 0.01 to 1×10\hat{ }4, 0.01 to 1000, 0.01 to 100, 0.01 to 10, 0.01 to 1, 0.01 to 0.1, 0.1 to 1×10\hat{ }4, 0.1 to 1000, 0.1 to 100, 0.1 to 10, 0.1 to 1, 1 to 1×10\hat{ }4, 1 to 1000, 1 to 100, or 1 to 10 viruses per leukocyte.

In some embodiments, adsorbing the myxoma virus onto the surface of the leukocytes comprises exposing the leukocytes to the MYXV at a multiplicity of infection (MOI) of between about 0.1 to 10. In some embodiments, adsorbing the myxoma virus onto the surface of the leukocytes comprises exposing the leukocytes to the MYXV at a multiplicity of infection (MOI) of between about 0.01 to 100. In some embodiments, adsorbing the myxoma virus onto the surface of the leukocytes comprises exposing the leukocytes to the MYXV at a multiplicity of infection (MOI) of between about 0.001 to 1000.

In some embodiments, the leukocytes are contacted or adsorbed with a MYXV of the disclosure for a period of about 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, 2.5 hours, 3 hours, 3.5 hours, 4 hours, 4.5 hours, 5 hours, 5.5 hours, 6 hours, 7 hours, 8 hours, 9 hours, 10 hours, 11 hours, 12 hours, 13 hours, 14 hours, 15 hours, 16 hours, 18 hours, 20 hours, 22 hours, or 24 hours.

In some embodiments, the leukocytes are contacted or adsorbed with a MYXV of the disclosure for a period of at least 5 minutes, at least 10 minutes, at least at least 15 minutes, at least 20 minutes, at least 25 minutes, at least 30 minutes, at least 35 minutes, at least 40 minutes, at least 45 minutes, at least 50 minutes, at least 55 minutes, at least 60 minutes, at least 65 minutes, at least 70 minutes, at least 75 minutes, at least 80 minutes, at least 85 minutes, at least 90 minutes, at least 95 minutes, at least 100 minutes, at least 105 minutes, at least 110 minutes, at least 115 minutes, at least 120 minutes, at least 2.5 hours, at least 3 hours, at least 3.5 hours, at least 4 hours, at least 4.5 hours, at least 5 hours, at least 5.5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 18 hours, at least 20 hours, at least 22 hours, at least 24 hours, or more.

In some embodiments, the leukocytes are contacted or adsorbed with a MYXV of the disclosure for a period of at most 5 minutes, at most 10 minutes, at most at most 15 minutes, at most 20 minutes, at most 25 minutes, at most 30 minutes, at most 35 minutes, at most 40 minutes, at most 45 minutes, at most 50 minutes, at most 55 minutes, at most 60 minutes, at most 65 minutes, at most 70 minutes, at most 75 minutes, at most 80 minutes, at most 85 minutes, at most 90 minutes, at most 95 minutes, at most 100 minutes, at most 105 minutes, at most 110 minutes, at most 115 minutes, at most 120 minutes, at most 2.5 hours, at most 3 hours, at most 3.5 hours, at most 4 hours, at most 4.5 hours, at most 5 hours, at most 5.5 hours, at most 6 hours, at most 7 hours, at most 8 hours, at most 9 hours, at most 10 hours, at most 11 hours, at most 12 hours, at most 13 hours, at most 14 hours, at most 15 hours, at most 16 hours, at most 18 hours, at most 20 hours, at most 22 hours, at most 24 hours, or less.

In some embodiments, the BM or PBMC cells are contacted or adsorbed with MYXV constructs ex vivo for about one hour.

### Additional Ex Vivo Methods

As disclosed herein, MYXV is capable of selectively infecting cells that have a deficient innate anti-viral response, and can be used as an indicator of such a deficiency in cells. Thus, cells removed from a subject may be assayed for deficiency in an innate anti-viral response using the methods of the present disclosure. Such determination may indicate, when combined with other indicators, that the subject may be suffering from a particular disease state, for example, cancer. The cells may be removed from a subject, including a human subject, using known biopsy methods. The biopsy method will depend on the location and type of cell that is to be tested. Cells can be cultured and exposed to MYXV, for example by adding live MYXV to the culture medium. The multiplicity of infection (MOI), may be varied to determine an appropriate MOI for a given cell type, density and culture technique, using a positive control cell culture that is known to be infected upon exposure to MYXV.

The amount of MYXV added to the cultured cells may be varied depending on cell type, method of culturing and strain of virus.

Infectivity of the cultured cells by MYXV may be determined by various methods known to a skilled person, including the ability of the MYXV to cause cell death. It may also involve the addition of reagents to the cell culture to complete an enzymatic or chemical reaction with a viral expression product. The viral expression product may be expressed from a reporter gene that has been inserted into the MYXV genome.

In one embodiment, the MYXV may be modified to enhance the ease of detection of infection state. For example, the MYXV may be genetically modified to express a marker that can be readily detected by phase contrast microscopy, fluorescence microscopy, or by radioimaging. The marker may be an expressed fluorescent protein or an expressed enzyme that may be involved in a colorimetric or radiolabeling reaction. In some embodiments, the marker may be a gene product that interrupts or inhibits a particular function of the cells being tested.

### Pharmaceutical Compositions

An MYXV of the disclosure or a cell comprising an MYXV of the disclosure can be formulated as an ingredient in a pharmaceutical composition. Therefore, in some embodiments, the disclosure provides a pharmaceutical composition comprising a Myxoma virus expressing one or more immunomodulatory transgenes, and a pharmaceutically acceptable diluent or excipient. The compositions may contain pharmaceutically acceptable concentrations of salt, buffering agents, preservatives and various compatible carriers.

The pharmaceutical compositions may contain additional therapeutic agents, such as additional anti-cancer agents. In one embodiment, the compositions include a chemotherapeutic agent. The chemotherapeutic agent, for example, may be substantially any agent, which exhibits an effect against cancer cells or neoplastic cells of the subject and that does not inhibit or diminish the tumor killing effect of the MYXV expressing one or more immunomodulatory transgenes. For example, the chemotherapeutic agent may be, without limitation, an anthracycline, an alkylating agent, an alkyl sulfonate, an aziridine, an ethylenimine, a methylmelamine, a nitrogen mustard, a nitrosourea, an antibiotic, an antimetabolite, a folic acid analogue, a purine analogue, a pyrimidine analogue, an enzyme, a podophyllotoxin, a platinum-containing agent or a cytokine. The chemotherapeutic agent can be one that is known to be effective against the particular cell type that is cancerous or neoplastic.

The proportion and identity of the pharmaceutically acceptable diluent can be determined, for example, by chosen route of administration, compatibility with a live virus, and standard pharmaceutical practice. In some embodiments, the pharmaceutical composition will be formulated with components that will not significantly impair the biological properties of the MYXV expressing one or more immunomodulatory transgenes. The pharmaceutical composition can be prepared by known methods for the preparation of pharmaceutically acceptable compositions suitable for administration to subjects, such that an effective quantity of the active substance or substances is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1995). On this basis, the compositions can comprise solutions of the MYXV or cells comprising the MYXV in association with one or more pharmaceutically acceptable vehicles or diluents, and contained in buffer solutions with a suitable pH and iso-osmotic with physiological fluids.

The pharmaceutical composition may be administered to a subject in a variety of forms depending on the selected route of administration, as disclosed herein. The composition of the invention may be administered orally or parenterally. Parenteral administration includes intravenous, intraperitoneal, subcutaneous, intramuscular, transepithelial, nasal, intrapulmonary, intrathecal, intratumoral, rectal, and topical modes of administration. Parenteral administration may be by continuous infusion over a selected period of time (e.g., intravenous infusion).

The pharmaceutical composition may be administered orally, for example, with an inert diluent or with a carrier, or it may be enclosed in hard or soft shell gelatin capsules, or it may be compressed into tablets. For oral therapeutic administration, the MYXV expressing one or more immunomodulatory transgenes may be incorporated with an excipient and be used in the form of ingestible tablets, buccal tablets, troches, capsules, elixirs, suspensions, syrups, wafers and the like.

Solutions of an MYXV of the disclosure or cells comprising an MYXV of the disclosure can be prepared in a physiologically suitable buffer. Under ordinary conditions of storage and use, these preparations can contain a preservative to prevent the growth of microorganisms, but that will not inactivate the live virus. Conventional procedures and ingredients for the selection and preparation of suitable formulations are described, for example, in Remington's Pharmaceutical Sciences and in The United States Pharmacopeia: The National Formulary (USP 24 NF 19) published in 1999. The dose of the pharmaceutical composition that is to be used depends on the particular condition being treated, the severity of the condition, the individual subject parameters including age, physical condition, size and weight, the duration of the treatment, the nature of concurrent therapy (if any), the specific route of administration and other similar factors that are within the knowledge and expertise of the health practitioner. In certain embodiments, the therapeutic virus may be freeze dried for storage at room temperature.

### Kits

Aspects of the present disclosure concern a MYXV that expresses one or more immunomodulatory transgenes, and kits including the same. The MYXV expressing one or more immunomodulatory transgenes, or pharmaceutical compositions comprising the MYXV, can be packaged as a kit, for example, containing instructions for use of the MYXV. A kit can comprise any MYXV disclosed herein, for example, a MYXV comprising one or more immunomodulatory transgenes, one or more reporter transgenes, one or more non-immunomodulatory transgenes, or a combination thereof. The kit can comprise one or more pharmaceutically-acceptable buffers, diluents, carriers, excipients, or vehicles, for example, for formulating the MYXV into a dosage form for administration to a recipient subject.

Disclosed herein, in some embodiments, is a kit that comprises a MYXV of the disclosure (e.g., a MYXV that expresses one or more immunomodulatory transgenes), and materials for cellular delivery of MYXV as disclosed herein. The kit can comprise, for example, a plurality of cells, such as leukocytes from bone marrow and/or peripheral blood. The leukocytes can be autologous, allogeneic, haploidentical, HLA-matched, or HLA-mismatched relative to a subject who will be a recipient of the MYXV and the cells. In some embodiments, the plurality of cells is pre-adsorbed with or have been exposed to the MYXV of the disclosure. The kit can comprise instructions for adsorbing the MYXV to the plurality of cells and/or administering the MYXV-adsorbed cells to a recipient. The kit can comprise one or more pharmaceutically-acceptable buffers, diluents, carriers, excipients, or vehicles, for example, for adsorbing the MYXV to the plurality of cells, removing unbound MYXV, formulating the MYXV-adsorbed cells into a dosage form for administration to a recipient subject, or any combination thereof.

In some embodiments, the kit comprises a MYXV of the disclosure and a plurality of cells. In some embodiments, the kit comprises a MYXV of the disclosure and instructions for adsorbing the MYXV to the plurality of cells and/or administering the MYXV-adsorbed cells to a recipient. In some embodiments, the kit comprises a MYXV of the disclosure and one or more pharmaceutically-acceptable buffers, diluents, carriers, excipients, or vehicles. In some embodiments, the kit comprises a MYXV of the disclosure, a plurality of cells, and instructions for adsorbing the MYXV to the plurality of cells and/or administering the MYXV-adsorbed cells to a recipient. In some embodiments, the kit comprises a MYXV of the disclosure, a plurality of cells, and one or more pharmaceutically-acceptable buffers, diluents, carriers, excipients, or vehicles. In some embodiments, the kit comprises a MYXV of the disclosure, instructions for adsorbing the MYXV to the plurality of cells and/or administering the MYXV-adsorbed cells to a recipient, and one or more pharmaceutically-acceptable buffers, diluents, carriers, excipients, or vehicles. In some embodiments, the kit comprises a plurality of cells, instructions for adsorbing a MYXV to the plurality of cells and/or administering the MYXV-adsorbed cells to a recipient, and one or more pharmaceutically-acceptable buffers, diluents, carriers, excipients, or vehicles. In some embodiments, the kit comprises a MYXV of the disclosure, a plurality of cells, instructions for adsorbing the MYXV to the plurality of cells and/or administering the MYXV-adsorbed cells to a recipient, and one or more pharmaceutically-acceptable buffers, diluents, carriers, excipients, or vehicles.

### EXAMPLES

### Example 1: Design of and Construction of Recombinant MYXV Constructs Expressing Immunomodulatory Transgenes

This example demonstrates the design and construction of recombinant MYXV constructs that express one or more immunomodulatory transgenes.

A DNA sequence encoding an immunomodulatory transgene of the disclosure is generated, e.g., obtained commercially or by PCR amplification. In some cases, the protein encoded can comprise a signal sequence to promote secretion. In some cases, the protein can comprise an epitope tag for detection and/or purification of the protein (e.g., a V5 tag).

A plasmid is generated for integration of the transgene into the myxoma virus genome. The transgene can be expressed under a poxvirus synthetic early/late promoter (sE/L), that allows expression only in virus-infected cells. In addition to transgene, a reporter gene, for example green fluorescent protein (GFP) or TdTomato, can also be optionally included and expressed under a poxvirus promoter for quick selection and purification of transgene-expressing recombinant virus. Additional reporter genes, for example Firefly luciferase (F-Luc) can allow real time monitoring of viral replication, e.g., in live animals. The transgene and reporter genes can be inserted between open reading frames to maintain a parental wild type MYXV backbone (for example, between the open reading frames for M135 and M136. Transgenes can also be inserted in a gene knockout virus background. For example, a transgene be inserted in the M135 locus, with a corresponding deletion or disruption of the M135 gene, or a transgene can be inserted in the M136 locus, with a corresponding deletion or disruption of the M136 gene.

The final recombinant plasmid cassette contains: (i) transgene, (ii) optional reporter gene(s), and (iii) homology arms/recombination arms with DNA sequences that match sequences in the virus genome to facilitate homologous recombination.

The construction of the recombination plasmids is done by Gateway technology (Multisite Gateway Pro), which allows construction of a single plasmid from multiple DNA fragments by recombination in bacteria. Multiple entry clones are generated containing different elements to make the final recombination cassette. The entry clones can contain: (i) a first homology/recombination arm comprising a DNA sequence that matches a sequence in the virus genome; (ii) an immunomodulatory transgene of the disclosure with a promoter (e.g., poxvirus synthetic early/late promoter), optionally with a signal sequence, an epitope tag, etc.; (iii) optionally, one or more reporter genes with promoters (e.g., GFP and/or TdTomato under control of a poxvirus late p11 promoter); and (iv) a second homology/recombination arm comprising a DNA sequence that matches a sequence in the virus genome. For constructing the final recombination plasmid cassette, all the elements are recombined with a Gateway destination vector by LR recombination reaction using a standard protocol.

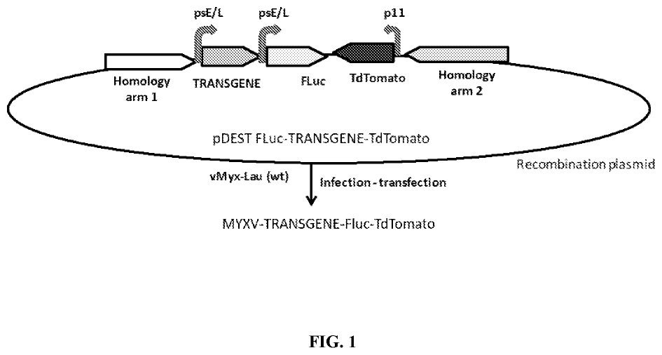

Examples of recombination plasmids are provided in FIG. 1. The two homology arms can align to sequences that converge (e.g., for insertion of the recombination cassette in at an intergenic region). In some cases, homology arm 1 can comprise the M135 open reading frame and homology arm 2 can comprise the M136 open reading frame, facilitating insertion of a transgene and reporter gene(s) in between the M135 and M136 open reading frames of the myxoma virus genome.

The two homology arms can align to two separate sequences in the viral genome that skip over a sequence to be deleted (e.g., for deletion of all or part of a gene of the viral genome, and replacement with recombination cassette). In some cases, homology arm 1 can comprise the M134 open reading frame or part thereof, and none or a fragment of the M135 open reading frame, and homology arm 2 can comprise the M136 open reading frame or a part thereof, and none or a fragment of the M135 open reading frame, so that the rest of the M135 open reading frame can be deleted. In some cases, homology arm 1 can comprise a sequence that is upstream of SOD, and none or a fragment of the SOD open reading frame, and homology arm 2 can comprise a sequence that is downstream of the SOD open reading frame or a part thereof, and none or a fragment of the SOD open reading frame, so that the rest of the SOD open reading frame can be deleted.

After plasmid construction, expression of the transgene(s) and/or reporter gene(s) can be tested by Western blot analysis after transfection of the plasmids into MYXV-infected RK13 cells.

The final recombination plasmids encoding the transgenes and reporter genes together with the flanking sequences are transfected into RK13 cells that are infected with wild type MYXV Lausanne. Recombinant viruses are isolated and serially purified based on the expression of reporter genes marker.

Expression of the immunomodulatory transgenes is again confirmed by Western blot analysis and functional assays. Viruses are generated comprising the immunomodulatory transgenes on a wild type virus background, and on a knockout background (e.g., with knockout of M135).

This method is used to generate MYXV expressing immunomodulatory transgenes of the disclosure, such as STING, TNFα, anti-PDL1, FAST, and/or IL-12.

### Example 2: Design and Construction of a Recombinant MYXV that Expresses STING

This example demonstrates the design and construction of a recombinant MYXV that expresses a constitutively activated variant of STING. The version of the STING utilized gene has a gain-of-function mutation (R284S), which is due to the substitution of a single base pair (852G/T). The mutant cDNA STING was synthetized by GenScript, comprising the sequence of SEQ ID NO: 3. The 852G/T mutation is indicated by underlining and bold.

SEQ ID NO: 3:
ATGCCCCACTCCAGCCTGCATCCATCCATCCCGTGTCCCA
GGGGTCACGGGGCCCAGAAGGCAGCCTTGGTTCTGCTGA
GTGCCTGCCTGGTGACCCTTTGGGGGCTAGGAGAGCCACC
AGAGCACACTCTCCGGTACCTGGTGCTCCACCTAGCCTCC
CTGCAGCTGGGACTGCTGTTAAACGGGGTCTGCAGCCTGG
CTGAGGAGCTGCGCCACATCCACTCCAGGTACCGGGGCAG
CTACTGGAGGACTGTGCGGGCCTGCCTGGGCTGCCCCCTC
CGCCGTGGGGCCCTGTTGCTGCTGTCCATCTATTTCTACT
ACTCCCTCCCAAATGCGGTCGGCCCGCCCTTCACTTGGAT
GCTTGCCCTCCTGGGCCTCTCGCAGGCACTGAACATCCTC
CTGGGCCTCAAGGGCCTGGCCCCAGCTGAGATCTCTGCAG
TGTGTGAAAAAGGGAATTTCAACGTGGCCCATGGGCTGGC
ATGGTCATATTACATCGGATATCTGCGGCTGATCCTGCCA
GAGCTCCAGGCCCGGATTCGAACTTACAATCAGCATTACA
ACAACCTGCTACGGGGTGCAGTGAGCCAGCGGCTGTATAT
TCTCCTCCCATTGGACTGTGGGGTGCCTGATAACCTGAGT
ATGGCTGACCCCAACATTCGCTTCCTGGATAAACTGCCCC
AGCAGACCGGTGACCATGCTGGCATCAAGGATCGGGTTTA
CAGCAACAGCATCTATGAGCTTCTGGAGAACGGGCAGCGG
GCGGGCACCTGTGTCCTGGAGTACGCCACCCCCTTGCAGA
CTTTGTTTGCCATGTCACAATACAGTCAAGCTGGCTTTAG
CCGGGAGGATAGTCTTGAGCAGGCCAAACTCTTCTGCCGG
ACACTTGAGGACATCCTGGCAGATGCCCCTGAGTCTCAGA
ACAACTGCCGCCTCATTGCCTACCAGGAACCTGCAGATGA
CAGCAGCTTCTCGCTGTCCCAGGAGGTTCTCCGGCACCTG
CGGCAGGAGGAAAAGGAAGAGGTTACTGTGGGCAGCTTGA
AGACCTCAGCGGTGCCCAGTACCTCCACGATGTCCCAAGA
GCCTGAGCTCCTCATCAGTGGAATGGAAAAGCCCCTCCCT
CTCCGCACGGATTTCTCTTGA

MYXV-STING was constructed by inserting a STING-expressing cassette with a C-terminal V5-tag at the M135 locus in the wild-type (wt) MYXV strain Laussane (MYXV-Lau) genome. STING expression is under the control of the poxvirus P11 late promoter (P11). An expression cassette for an enhanced green fluorescent protein (eGFP) was inserted immediately downstream of the STING, and its expression was driven by a poxvirus synthetic early/late promoter (sE/L). The eGFP can serve as a fluorescent marker for MYXV replication in vitro and in vivo, as MYXV infection can be monitored by live imaging of GFP expression.

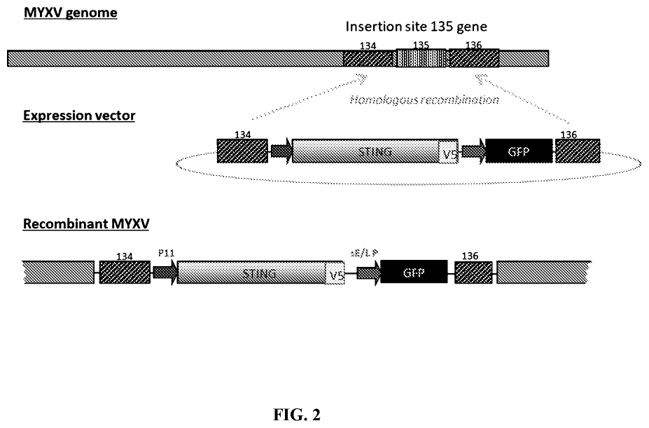

To create the MYXV-STING-GFP construct, a recombination plasmid was constructed using the Gateway System (ThermoFisher Scientific). FIG. 2 illustrates the design of the recombination plasmid, and the modified genome of MYXV-STING-GFP after recombination.

Upstream and downstream hybridizing sequences were amplified by PCR to generate entry clones by Gateway BP recombination with appropriate pDONR vectors. The final recombinant plasmid was constructed by recombining three entry clones with a destination vector in a sequential manner. The STING and eGFP expression cassettes were inserted into the MYXV genome by infecting RK13 cells with MYXV-Lau and then transfecting the appropriate recombination plasmid. Multiple rounds of foci purification were conducted to obtain pure stocks of the recombinant viruses, the specificity confirmed by PCR using the appropriate primers set:

AttB4r_hSTING_F (SEQ ID NO: 30):
5′GGGGACAACTTTTCTATACAAAGTTGC
CGAATTTCATTTTGTTTTTTTCTATGCTA
TAA ATGCCCCACTCCAGCCTGCATCC-3′
AttB3r_hSTING_R (SEQ ID NO: 31):
5′GGGACAACTTTATTATACAAAGTTGTTT
AAGAATCGAGACCGAGGAGAGGGTTAGGGA
TAGGCTTACC AGAGAAATCCGTGCGGAGA
G-3′

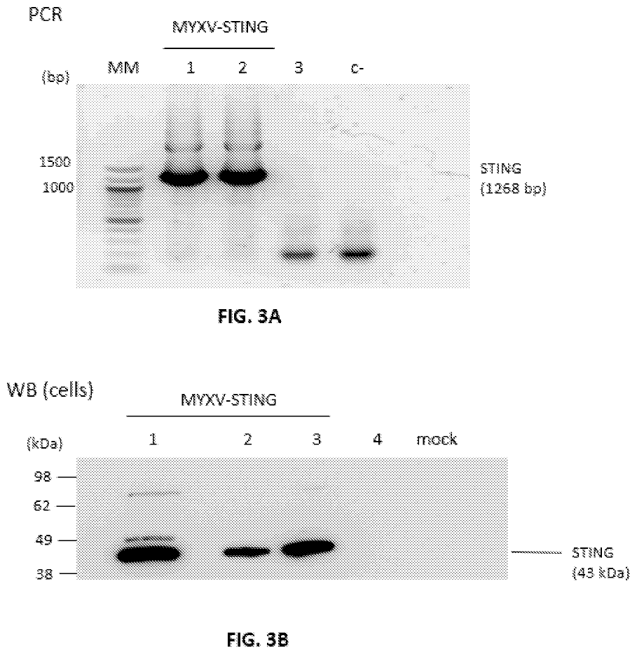

FIG. 3A illustrates detection of STING via agarose gel electrophoresis of PCR products. Candidates 1 and 2 of FIG. 3A are positive for STING. Candidate 3 and a control are negative for STING.

STING protein expression was confirmed by Western Blot of lysates from MYXV-STING-GFP infected RK13 cells. A mouse monoclonal antibody specific to the V5 epitope tag was used, allowing detection of a band of 43 kDa (FIG. 3B). STING was detected in lysates from three of the RK13 cell cultures, while a fourth culture and a control were negative.

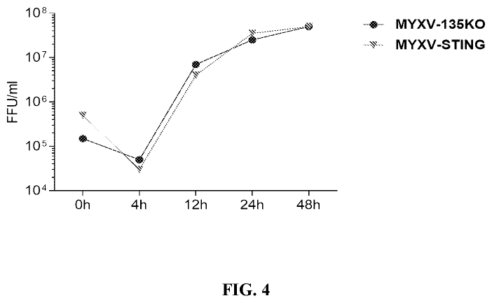

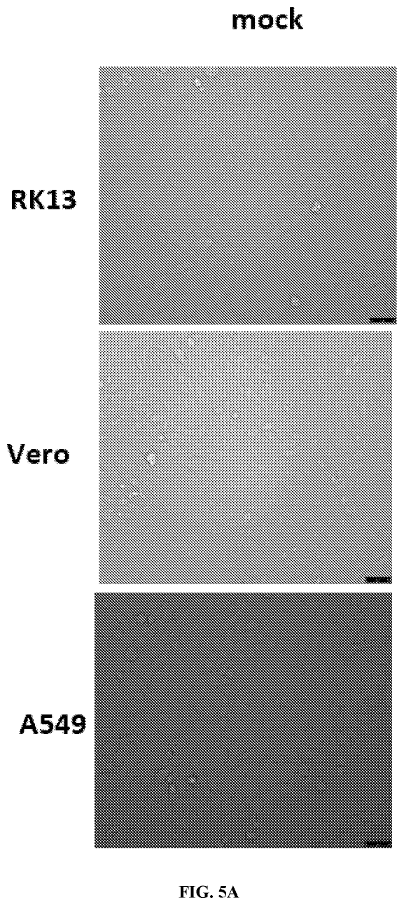

The replication capacity of MYXV-STING was tested via a single step growth curve in RK13 cells infected at an MOI of 5. Replication was similar between MYXV-STING and a control parental virus (FIG. 4).

### Example 3: MYXV-STING Induces Autophagy in Infected Cells

During purification of MYXV-STING-GFP from the parental virus, it was unexpectedly observed that this new virus promoted a particular phenotype in the RK13-infected cells, characterized by the presence of hole-like, or vacuolar-like structures in infected cells.

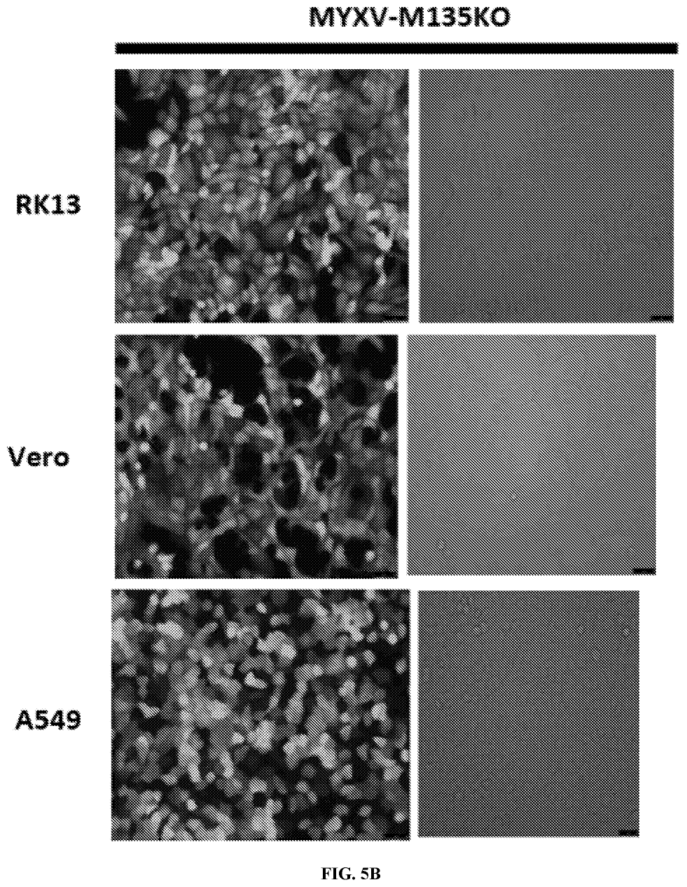

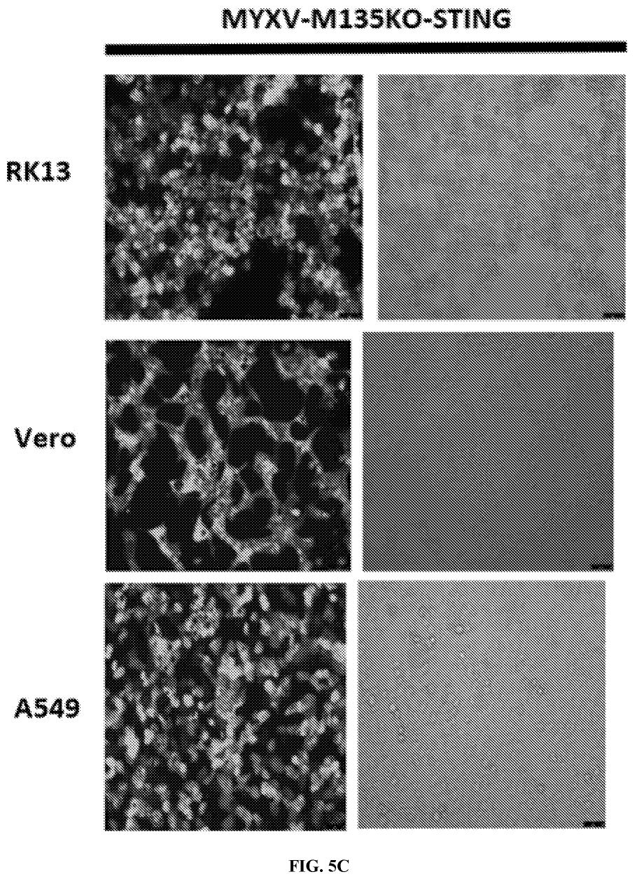

Purified MYXV-STING was used to infect RK13, Vero, and A549 cell lines at a multiplicity of infection (MOI) of 10. After 48 hours, all three cell lines exhibited the hole-like or vacuolar-like structures. FIG. 5A provides light microscopy images of mock-infected RK13, Vero, and A549 cells. FIG. 5B provides fluorescence and light microscopy images of RK13, Vero, and A549 cells infected with MYXV-M135KO-GFP. FIG. 5C provides fluorescence and light microscopy images of RK13, Vero, and A549 cells infected with MYXV-M135KO-STING-GFP.

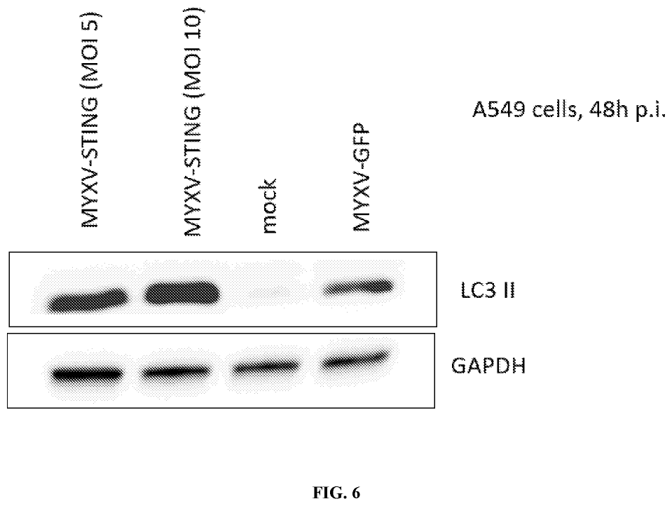

One possible explanation for the observed phenotype was the induction of autophagy in infected cells, which can lead to the formation of autophagosomes. To investigate whether the autophagy was induced in MYXV-STING-infected cells, A549 cells were infected with MYXV or MYXV-STING, and Western Blots were performed on cell lysates from 48 hours post-infection to detect LC3-II. During autophagy, LC3-I is converted to LC3-II through lipidation by a ubiquitin-like system involving Atg7 and Atg3 that allows for LC3 to become associated with autophagic vesicles. The presence of LC3 in autophagosomes and the conversion of LC3 to the lower migrating form, LC3-II, have been used as indicators of autophagy. The rabbit anti human-LC3B Ab (2775S) was used to detect LC3-II. The reference gene GAPDH was used as a loading control. The presence of STING in the viral genome appeared to upregulate LC3-II in a MOI dependent manner (FIG. 6). Surprisingly, MYXV-GFP also up-regulated LC3-II to a degree, indicating some degree of autophagy induction in infected cell cultures (FIG. 6).

### Example 4: MYXV Expressing Immunomodulatory Transgenes Infect and Kill Human Hematologic Cancer Cells In Vitro

In order to evaluate the susceptibility of human hematologic cancer cells to MYXV of the disclosure, human acute myeloid leukemia (AML) and multiple myeloma (MM) cell lines were infected with recombinant MYXV clones. THP-1 cells were used as an example of AML cells. U266 cells were used as an example of MM cells. U266 cells were maintained in RPMI 1640 supplemented with 20% fetal bovine serum (FBS), 2 mM L-Glutamine, and 100 U/ml of penicillin-streptomycin. THP-1 cells were maintained in RPMI 1640 supplemented with 10% FBS, 2 mM L-Glutamine, and 100 U/ml of penicillin-streptomycin.

Cells were mock-infected, or infected with MYXV-WT-GFP, MYXV-M135KO-GFP, MYXV-SODKO-TNFα-GFP, MYXV-anti-muPDL1-GFP, or MYXV-p14FAST-GFP at a multiplicity of infection (MOI) of 0.1, 1, or 10. Cells were infected at 37° C. for 1 hour to allow virus adsorption, then incubated to 24 or 48 hours post infection (hpi).

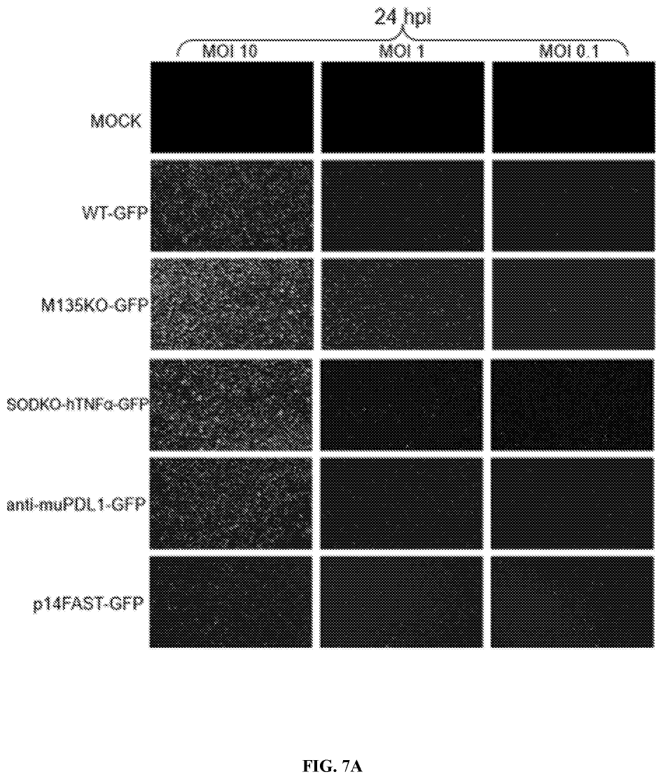

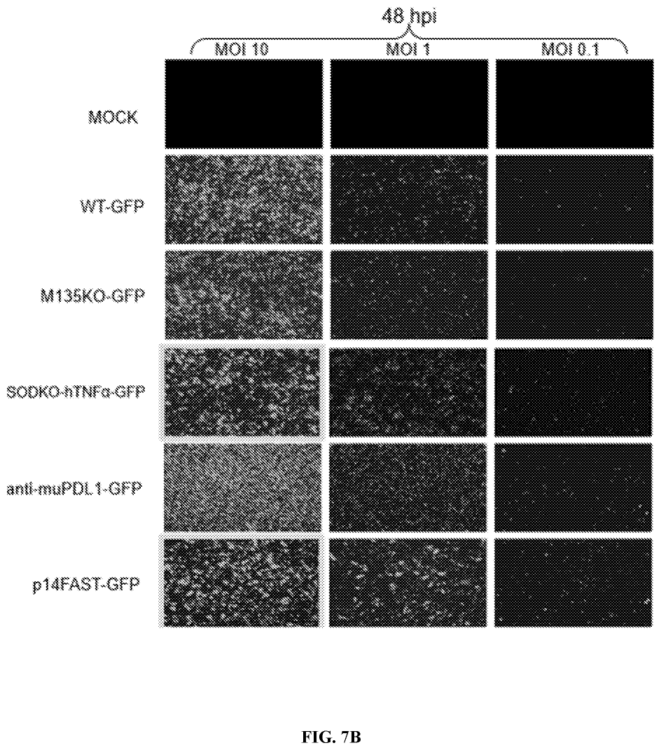

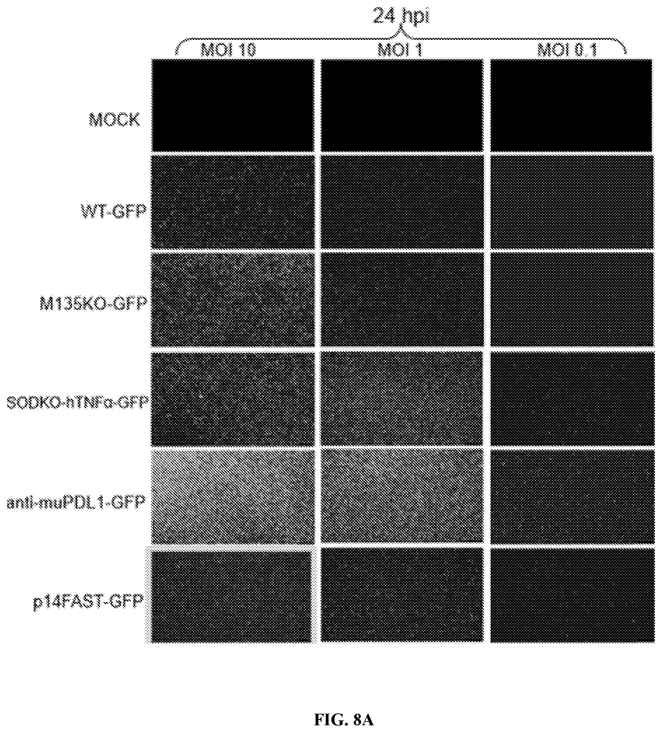

Infection was evaluated at 24 and 48 hpi by fluorescence microscopy. Images were taken at 5× magnification, with 338.00 ms exposure, and 2.5 gain. FIG. 7A and FIG. 7B demonstrate infection of THP-1 cells at 24 and 48 hours post-infection, respectively. FIG. 8A and FIG. 8B demonstrate infection of U266 cells at 24 and 48 hours post-infection, respectively.

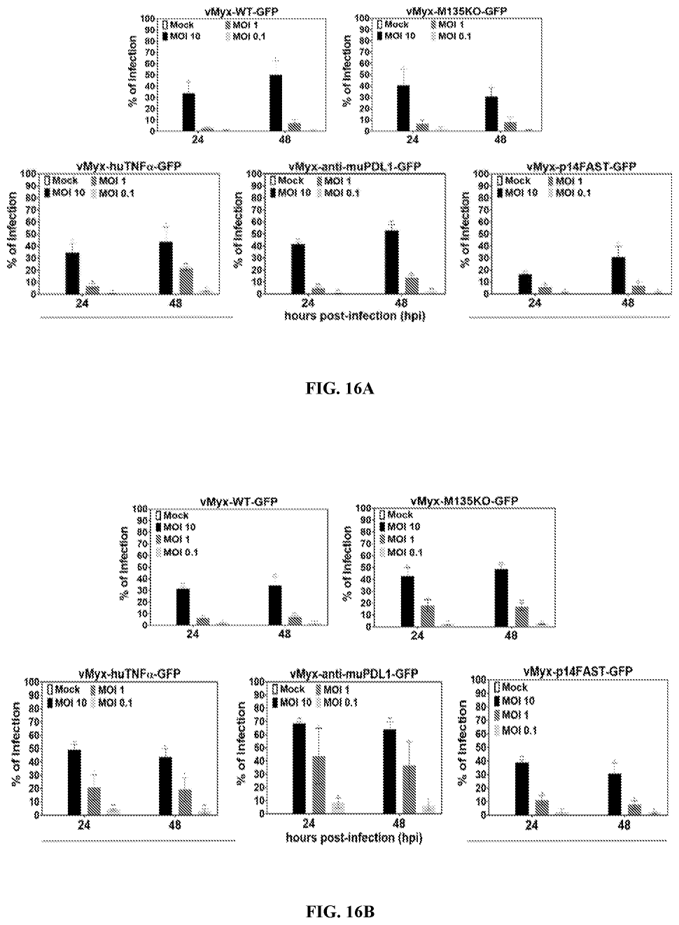

The infection rate was also quantified by flow cytometry, with populations of infected cells evaluated for GFP expression. FIG. 16A shows the percent of THP-1 cells that were GFP positive at 24 and 48 hours post-infection. FIG. 16B shows the percent of U266 cells that were GFP positive at 24 and 48 hours post-infection.

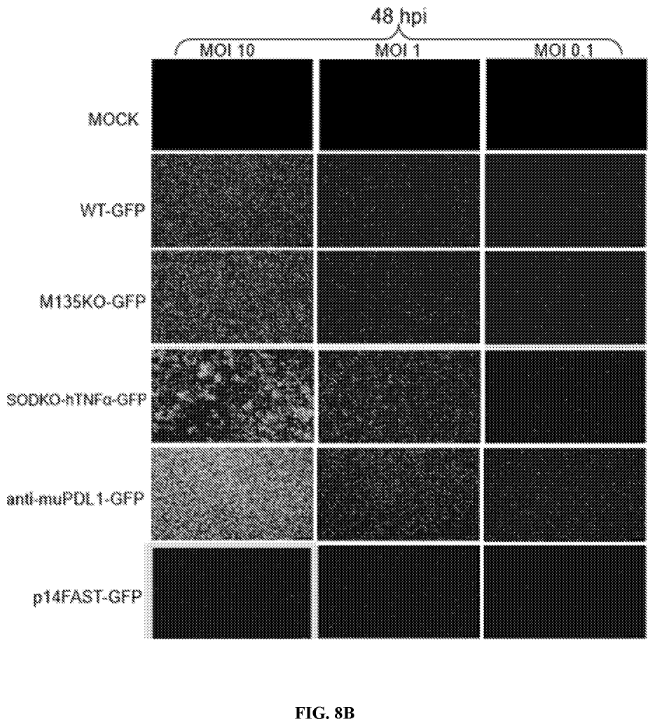

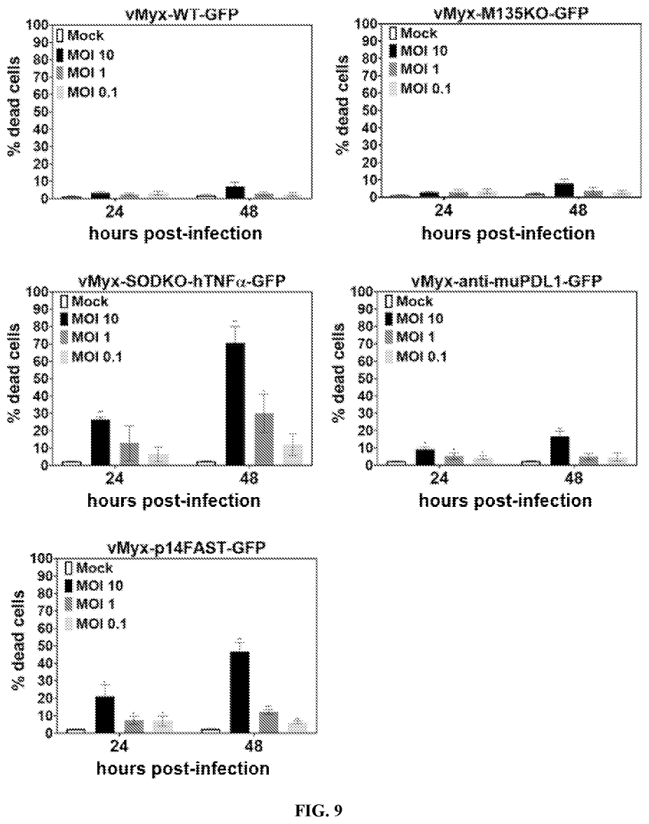

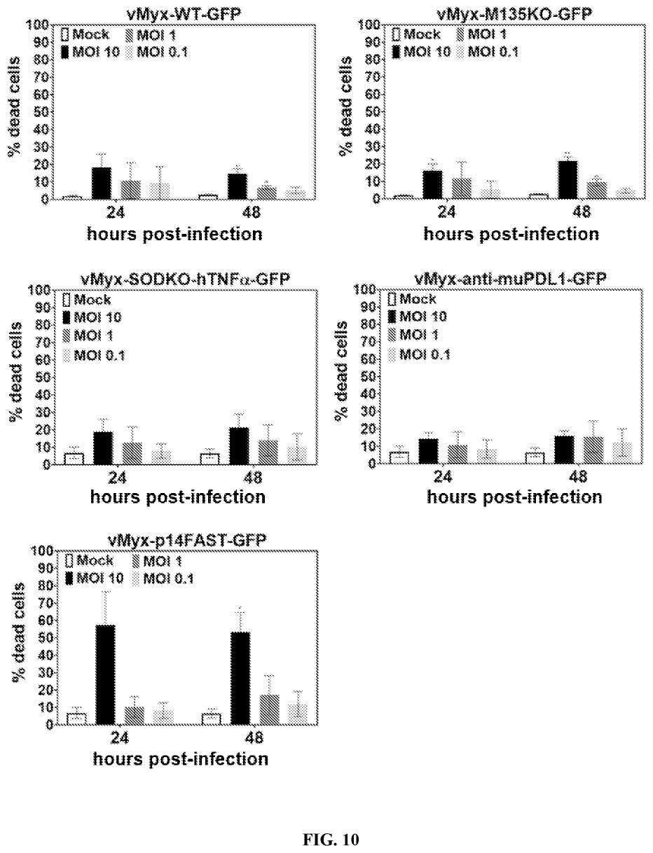

Cell killing was evaluated at 24 and 48 hpi by flow cytometry using a near-IR live/dead stain. FIG. 9 demonstrates killing of THP-1 cells. FIG. 10 demonstrates killing of U266 cells.

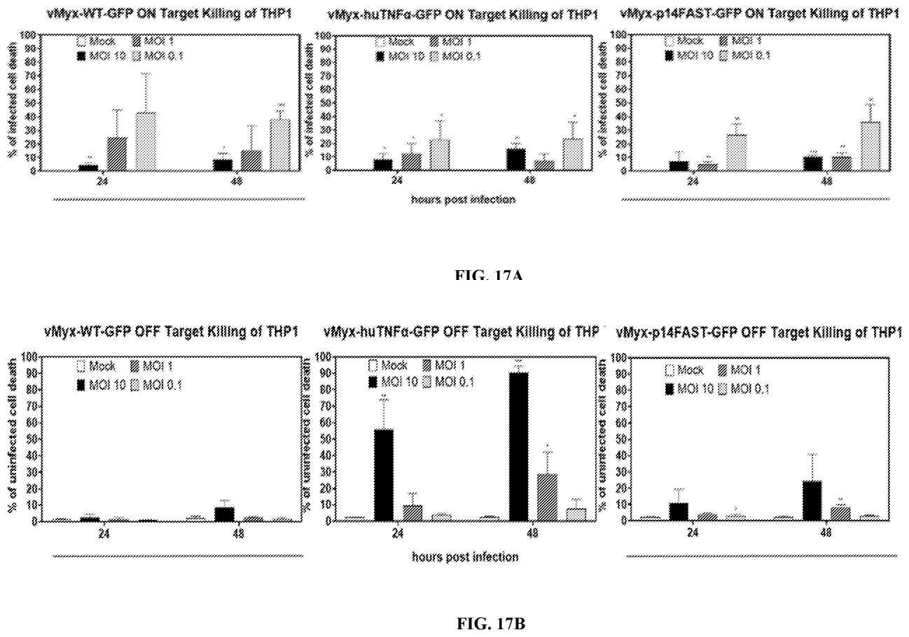

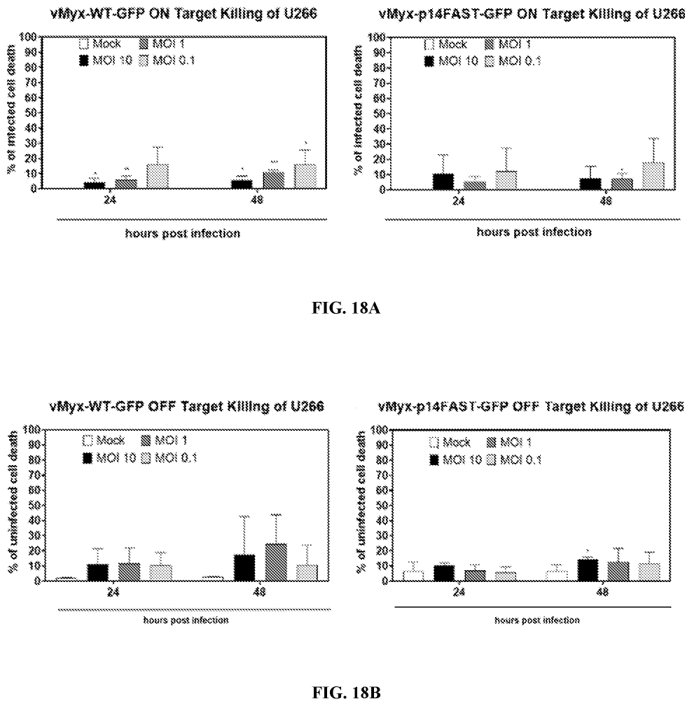

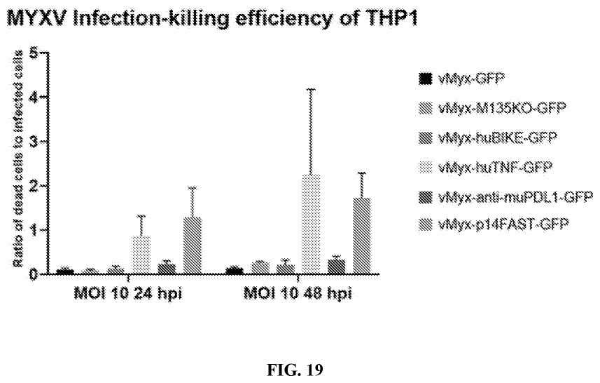

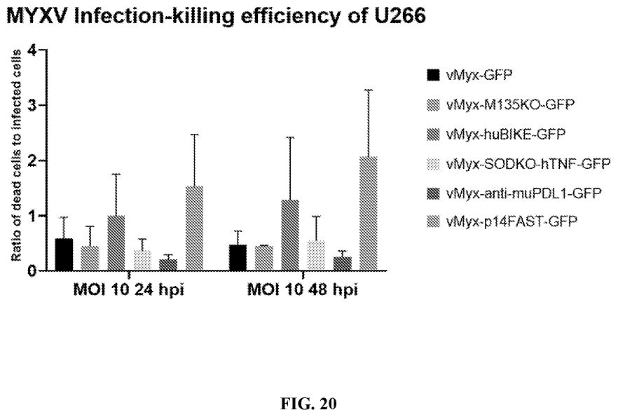

Cell killing was further characterized by gating GFP+ cells (for direct killing of infected cells, or on-target killing) and GFP negative cells (for indirect killing of uninfected cells, or off-target killing). FIG. 17A illustrates the percent of infected THP-1 cells that were killed at 24 and 48 hours. FIG. 17B illustrates the percent of uninfected THP-1 cells that were killed at 24 and 48 hours. FIG. 18A illustrates the percent of infected U266 cells that were killed at 24 and 48 hours. FIG. 18B illustrates the percent of uninfected U266 cells that were killed at 24 and 48 hours. FIG. 19 provides the ratio of dead THP-1 cells to infected THP-1 cells. FIG. 20 provides the ratio of dead U266 cells to infected U266 cells.

These data demonstrate that MYXV expressing immunomodulatory transgenes of the disclosure can infect, replicate within, and kill human hematologic cancer cells, and, in some cases, can elicit enhanced killing compared to MYXV lacking immunomodulatory transgenes. For example, MYXV-TNFα and MYXV-FAST elicited enhanced killing of THP-1 (human AML) cells, and MYXV-FAST elicited enhanced killing of U266 (human MM) cells compared to wild-type MYXV.

### Example 5: MYXV Expressing Immunomodulatory Transgenes Kill Primary Human Multiple Myeloma Cells from Bone Marrow

This example demonstrates MYXV of the disclosure killing multiple myeloma (MM) cells in bone marrow (BM) samples from human patients.

Primary bone marrow samples were obtained from multiple myeloma patients via bone marrow biopsy, and were subjected to purification using Ficoll-paque plus gradient to isolate mononuclear cells. Mononuclear cells were then resuspended in 380 μL complete media per condition into 24-well plates.

These primary cells in suspension were then mock-treated (i.e., no virus added), or incubated with MYXV-WT-GFP, MYXV-M135KO-GFP, MYXV-SODKO-TNFα-GFP, MYXV-anti-muPDL1-GFP, MYXV-p14FAST-GFP, MYXV-STING-GFP, or MYXV-IL12-GFP. Experiments were conducted at different multiplicities of infection (MOI) including MOI=10, 1, and 0.1. Cells were incubated at 37° C. for 1 hour to allow virus adsorption. After the 1 hour of incubation, 120 μL complete media was added to each well and the plates were left to further incubate at 37° C. At 24 hours post infection, the cells were then labeled with near-IR LIVE/DEAD. The primary cells were subsequently labeled with 1 μL human anti-CD138 antibody in 100 μL staining buffer per condition and incubated for 15 minutes at 4° C. with light protection. All samples were then fixed using 100 μL Cytofix, incubated for 15 minutes at 4° C. with light protection, then resuspended in 270 μL staining buffer for flow cytometry analysis.

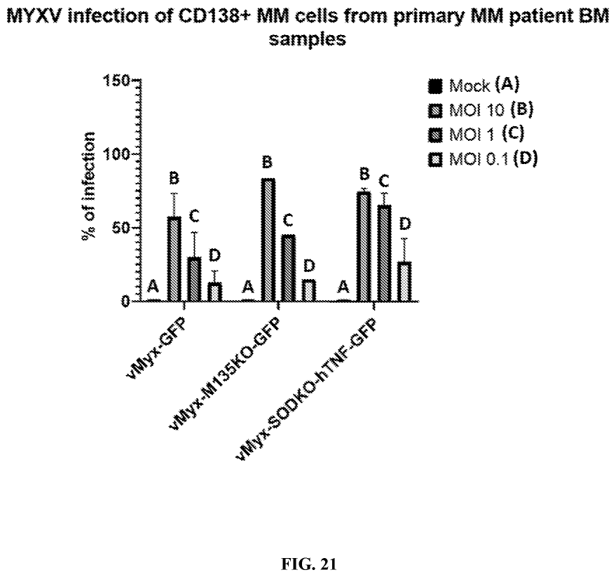

The percent of killed MM cells was evaluated by flow cytometry. CD138 was used as a marker of multiple myeloma (MM) cells. FIG. 21 shows the percent of CD138+ cells that were infected (GFP positive).

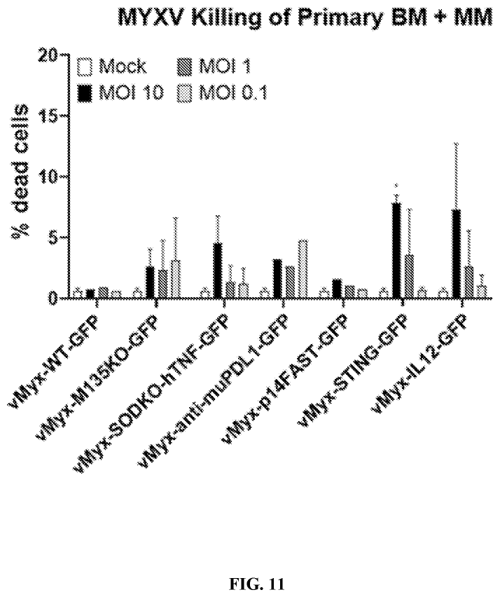

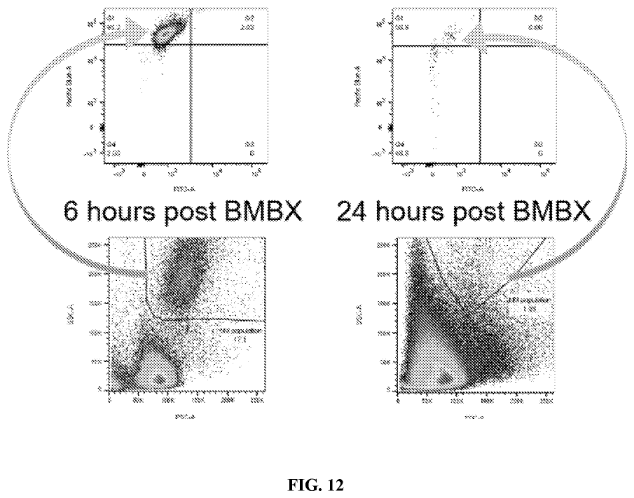

FIG. 11 demonstrates killing of MM cells by MYXV of the disclosure that express immunomodulatory transgenes. These data demonstrate that MYXV of the disclosure that express immunomodulatory transgenes can infect and kill primary human hematologic cancer cells, and, in some cases, can elicit enhanced killing compared to MYXV that does not express an immunomodulatory transgene. FIG. 12 shows a Pacific Blue 420-labeled MM cell population shown from the same primary cell sample, analyzed 6 and 24 hours post BM extraction during bone marrow biopsy, showing that these cells were present at 6 but were greatly reduced in number after 24 hours.

### Example 6: MYXV Expressing Immunomodulatory Transgenes Enhance Killing of Hematologic Cancer Cells

This example demonstrates evaluating a MYXV of the disclosure that expresses an immunomodulatory transgene for the ability to kill primary human hematologic cancer cells. Primary blood and bone marrow samples are obtained from patients with hematologic cancers. The samples are subjected to purification using Ficoll-paque plus gradient to isolate mononuclear cells and eliminate the majority of red blood cells (RBCs).

These primary cells in suspension are then mock-treated (i.e., no virus added), or incubated with MYXV of the disclosure at 37° C. for 1 hour to allow virus adsorption. Experiments are conducted at different multiplicities of infection (MOI) including MOI=10, 1, and 0.1. After this, mock-treated, or MYXV-treated cells are incubated overnight (˜24 hours) at 37° C. to allow virus infection to proceed.

The percentages of virus infection and the percentages of viability, apoptosis, and cell death of cancer cells are determined using flow cytometry. The percentages are also determined for uninfected cancer cells in patient samples that are exposed to the virus, allowing measurement of cancer cell death in cells that are not directly infected by the virus (e.g., do not express any virus-specific fluorescent protein), but are killed in an “off-target” fashion, e.g., killed by immune cells that are activated by the immunomodulatory transgene.

MYXV of the disclosure that express immunomodulatory transgenes (e.g., MYXV-SODKO-TNFα-GFP, MYXV-anti-muPDL1-GFP, MYXV-p14FAST-GFP, MYXV-STING-GFP, or MYXV-IL12-GFP) productively infect cancer cells. In some cases, MYXV of the disclosure that express immunomodulatory transgene directly kill cancer cells that they infect, and promote killing of un-infected cancer cells via the immunomodulatory transgene.

### Example 7: Oncolytic Virotherapy with Myxoma Virus (MYXV) Against Multiple Myeloma (MM): Identification of MYXV Constructs Suitable for Eliminating Cancer Cells from Primary Human Samples

Experiments are conducted to identify MYXV constructs and experimental conditions suitable for eliminating refractory cancer cells from primary human cell samples. Bone marrow or peripheral blood samples are obtained from a subject with a hematological cancer (e.g., a myeloma, a leukemia, or a lymphoma). Mononuclear cells are isolated (e.g., via Ficoll-Paque). Samples of mononuclear cells comprising cancer cells are treated with MYXV constructs of the disclosure (e.g., expressing one or more immunomodulatory transgenes and/or comprising one or more deletions) under various conditions (e.g., MOI, incubation time), and the ability of the MYXV constructs to kill cancer cells is determined as disclosed herein (e.g., via flow cytometry, fluorescence microscopy, and/or cytotoxicity assay).

The identified construct and/or experimental conditions can be used for treating the subject. For example, A MYXV construct identified as suitable can be directly administered to the subject (e.g., via injection or intravenous infusion), or can be administered via MYXV-adsorbed leukocytes.

### Example 8: Oncolytic Virotherapy with a Myxoma Virus (MYXV)

A subject is identified as having a hematological cancer (e.g., a myeloma, leukemia, or lymphoma). The hematological cancer can optionally be a hematological cancer that comprises minimal residual disease (MRD) and/or drug-resistant MRD.

Optionally, studies are conducted to identify a MYXV construct of the disclosure (e.g., expressing one or more immunomodulatory transgenes and/or comprising one or more deletions) that kills cancer cells from a sample taken from the subject (e.g., a peripheral blood or bone marrow sample).

A MYXV is administered to the subject (e.g., administered via injection or infusion). The MYXV infects cancer cells in the subject and expresses the immunomodulatory protein, leading to cancer cell killing and an enhanced anti-cancer immune response.

### Example 9: Oncolytic Virotherapy with Myxoma Virus (MYXV) Via Autologous Transplant of MYXV-Adsorbed Leukocytes

A MYXV is administered to a subject with a hematological cancer via autologous transplant of MYXV-adsorbed leukocytes.

Bone marrow or peripheral blood samples are obtained from a subject with a hematological cancer (e.g., a myeloma, leukemia, or lymphoma), and mononuclear cells are isolated (e.g., via Ficoll-Paque). Cancer cells can be separated from non-cancer cells (e.g., via FACS or MACS). A MYXV of the disclosure is adsorbed to leukocytes (for example, adsorbed for about an hour at an MOI of about 0.1 to 10). The MYXV-adsorbed leukocytes are administered back to the subject via intravenous infusion. The MYXV infects cancer cells in the subject, leading to cancer cell killing and an anti-cancer immune response.

### Example 10: Oncolytic Virotherapy with Myxoma Virus (MYXV) Via Allogenic Transplant of MYXV-Adsorbed Leukocytes

A MYXV is administered to a subject with a hematological cancer (e.g., a myeloma, leukemia, or lymphoma) via allogenic transplant of MYXV-adsorbed leukocytes. Bone marrow or peripheral blood samples are obtained from a donor (e.g., an HLA-matched, HLA-mismatched, haploidentical, or sibling donor, or a combination thereof). Mononuclear cells are isolated (e.g., via Ficoll-Paque). Optionally, cells are purified or enriched for specific leukocyte subsets (e.g., via FACS or MACS). A MYXV of the disclosure is adsorbed to leukocytes (for example, adsorbed for about an hour at an MOI of about 0.1 to 10). The MYXV-adsorbed leukocytes are administered back to the subject via intravenous infusion. The MYXV infects cancer cells in the subject, leading to cancer cell killing and an anti-cancer immune response.

### Example 11: Ex Vivo MYXV Virotherapy in Conjunction with Auto-Transplants in the Vk*MYC Immunocompetent Mouse Model of Minimal Residual Disease (MRD) to Target and Eliminate Drug-Resistant Disseminated MM In Vivo

This example demonstrates treatment of subjects with hematologic cancer by using MYXV adsorbed to the surface of leukocytes. Similar experiments or follow on experiments can be conducted with MYXV of the disclosure that express one or more immunomodulatory transgenes.

Two C57BL/6-derived VK*MYC cell lines were used for the in vivo experiments: VK12598, which is bortezomib-resistant (BOR-resistant), and the multi-drug resistant line VK12653. First, the susceptibility of these two VK*MYC cell lines to MYXV binding and infection was evaluated.

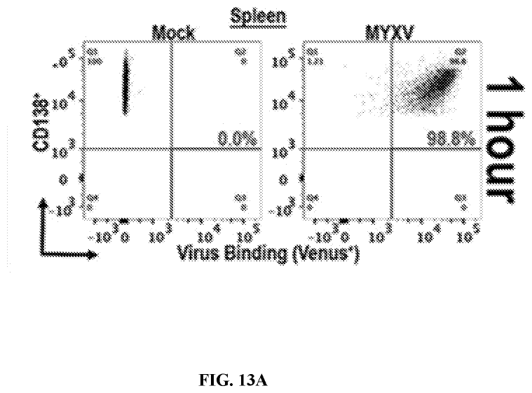

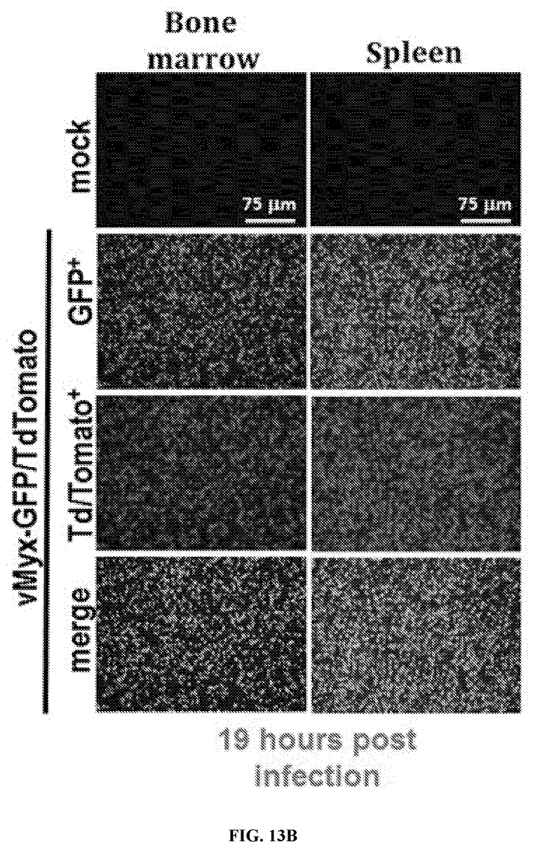

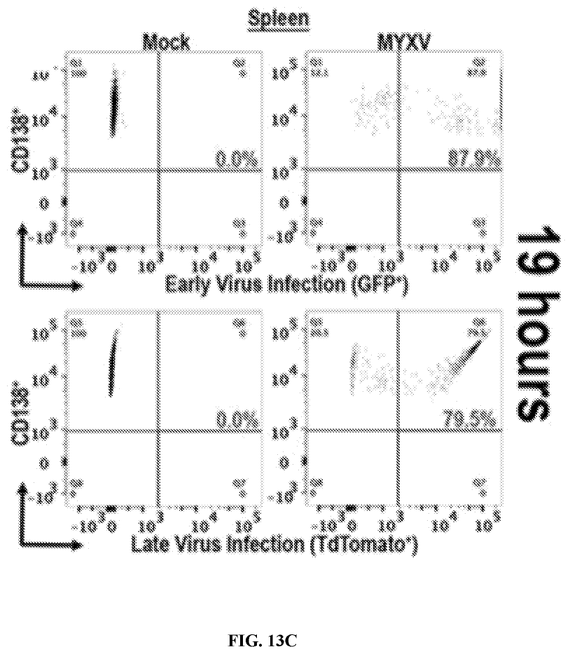

MYXV binding to VK12598 and VK12653, in vitro studies: For binding experiments, MYXV-M093L-Venus virus (comprising a fusion of the fluorescent protein Venus at the amino terminus of M093L) was used at a multiplicity of infection (MOI) of 10. In brief, either VK12598, or VK12653 were freshly isolated from BM (or from freshly-thawed BM), and incubated with MYXV-M093L-Venus at 4° C. for 1 hour to allow virus binding. Unbound virus was removed by washing the virus-adsorbed cells twice. Levels of virion binding were quantified using flow cytometry. For analyses of virus infection, cells were incubated with reporter MYXV-GFP(E/L)/TdTomato(L) at MOI=10 for 1 hour at 37° C. to allow virus adsorption. Cells were incubated overnight at 37° C. to allow virus infection. MYXV efficiently bound to both cell lines (FIGS. 13A and 14A). In addition to this, MYXV productively infects both cell lines (FIGS. 13B-C and 14B).

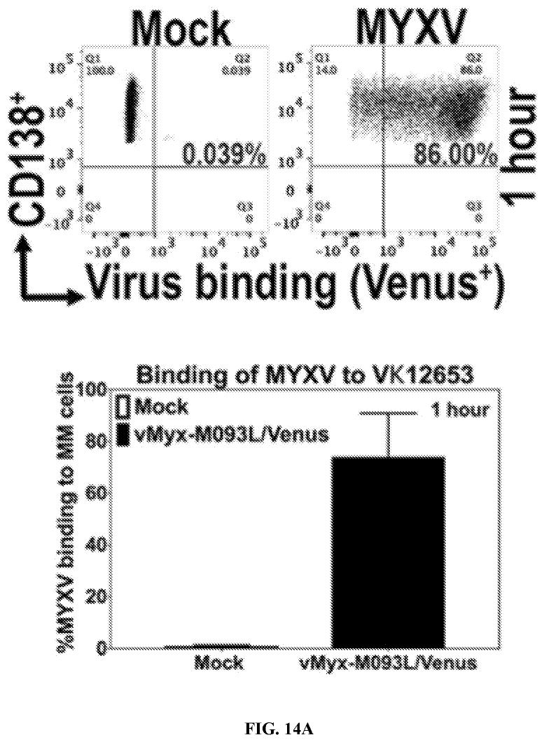

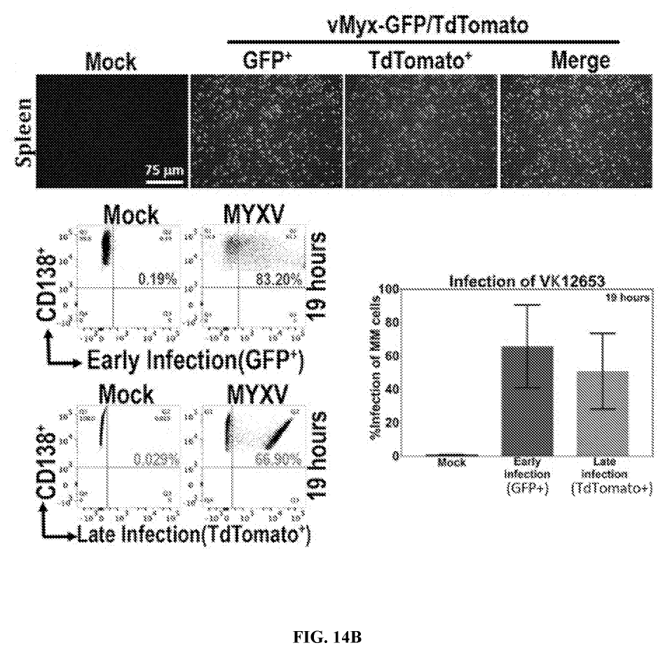

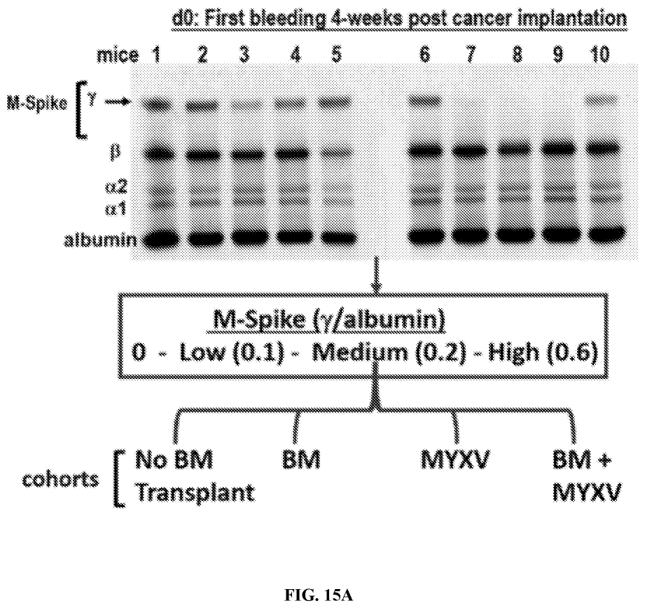

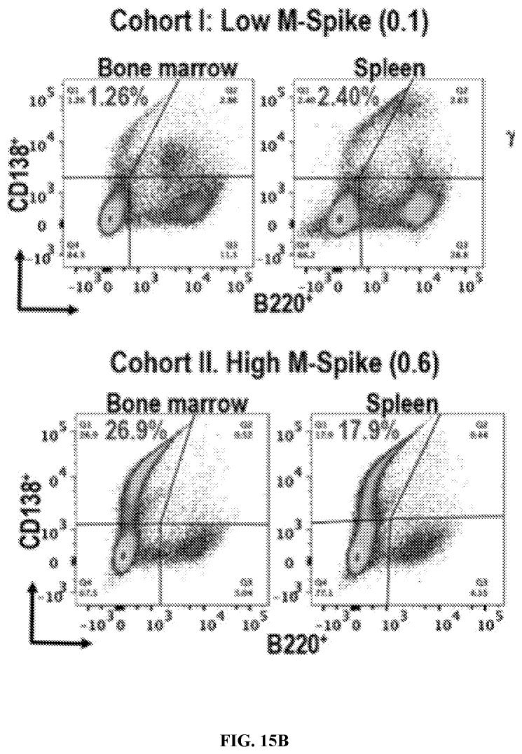

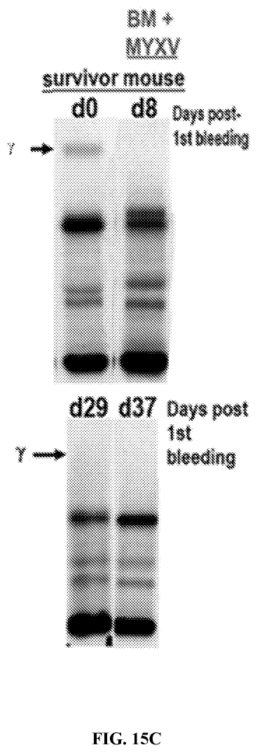

In vivo studies using the VK12598 cell line: In the first in vivo experiment, C57BL/6 mice were pre-seeded with VK12598 cells (e.g., 1×10⁶ cells per mouse). Four weeks post-MM cell implantation, mice were subjected to bleeding and the M-Spike was measured. Mice were separated according to the levels of M-Spike (e.g., 0, low=0.1, medium=0.2, high=0.6) (FIG. 15A, Top panel). Mice were then treated as follows: No C57BL/6 BM transplant (Cohort I), C57BL/6 BM cells alone (Cohort II), MYXV-M135KO-GFP alone (Cohort III), C57BL/6 BM ex vivo treated with MYXV-M135KO-GFP (Cohort IV) (FIG. 15A, bottom panel). FIG. 15B shows the percentage of MM (CD138⁺B220⁻) in a representative mouse from Cohort I with low M-spike (0.1) and the percentage of MM (CD138⁺B220⁻) in a representative mouse from Cohort II with high M-spike (0.6). FIG. 15C shows the M-spike of the only survivor from Cohort IV, which exhibited total regression of MM, with no M-spike band detected on day 8, day 29, and day 37 post-transplant. These data indicate that a transplant of ex vivo MYXV-treated bone marrow can induce MM regression. Together, these data may suggest that the cohort treatment in this first experiment started too late in the disease progression, and instead virotherapy should be started much earlier in this model (e.g. less than 1 week post-MM implantation rather than 4 weeks post-MM implantation). Although MM regression can occur even at this late intervention time, starting virotherapy earlier (e.g., in mouse cohorts that are not so close to death or end-point) may allow improved evaluation of the virus technology.

In additional trial, MYXV is tested in combination with other therapeutics (such as the SMAC mimetic LC161). VK12598 cancer cells are implanted, M-Spike quantified at 1-4 weeks, and the mice are treated with cyclophosphamide to induce a transient complete response (CR), which can last 1 month. At either one or two weeks post cyclophosphamide, the mice are transplanted with BM, MYXV, or PBMC+MYXV (e.g., MYXV expressing an immunomodulatory transgene as disclosed herein) in order to test if the virotherapy can extend or complete the partial regression initiated by the cyclophosphamide. In this setting, the capacity of MYXV to eliminate MM minimal residual disease (MRD) as defined by disease that functionally resists this chemotherapy is investigated. The capacity of MYXV to eliminate the multidrug-resistant VK12653 cells line, either as a monotherapy or in combination therapy is also investigated.

### EMBODIMENTS

Embodiment 1. A myxoma virus (MYXV) engineered to express one or more immunomodulatory proteins.

Embodiment 2. The myxoma virus of embodiment 1, wherein the immunomodulatory protein is Stimulator of interferon genes (STING).

Embodiment 3. The myxoma virus of embodiment 1, wherein the immunomodulatory protein is an interleukin-12 (IL-12).

Embodiment 4. The myxoma virus of embodiment 1, wherein the immunomodulatory protein is an IL-12A.

Embodiment 5. The myxoma virus of embodiment 1, wherein the immunomodulatory protein is an IL-12B.

Embodiment 6. The myxoma virus of embodiment 1, wherein the immunomodulatory protein is Fusion-associated small transmembrane (FAST).

Embodiment 7. The myxoma virus of embodiment 1, wherein the immunomodulatory protein is an immune checkpoint inhibitor.

Embodiment 8. The myxoma virus of embodiment 1, wherein the immunomodulatory protein is anti-PDL1.

Embodiment 9. The myxoma virus of embodiment 1, wherein the immunomodulatory protein is a tumor necrosis factor (TNF) protein.

Embodiment 10. The myxoma virus of any one of embodiments 1-9, wherein the immunomodulatory protein is capable of stimulating the toll like receptors (TLR) or activating Nuclear factor-κB (NF-κB) or (interferon regulatory factor) IRF.

Embodiment 11. The myxoma virus of any one of embodiments 1-10, wherein myxoma virus comprises a modification at or adjacent to M011 L, M063, M135R, M136R, M-T2, M-T4, M-T5, or M-T7.

Embodiment 12. A pharmaceutical composition comprising a myxoma virus of any one of embodiments 1-11, and a pharmaceutically acceptable excipient.

Embodiment 13. The pharmaceutical composition of embodiment 12, wherein the pharmaceutical composition is formulated for systemic administration.

Embodiment 14. The pharmaceutical composition of embodiment 13, wherein the pharmaceutical composition is formulated for local administration.

Embodiment 15. The pharmaceutical composition of any one of the embodiments 12-14, wherein the pharmaceutical composition is formulated for parenteral administration.

Embodiment 16. A composition comprising peripheral blood mononuclear cells (PBMCs), bone marrow (BM) cells, or a combination thereof treated ex vivo by a myxoma virus (MYXV) engineered to express an immunomodulatory protein.

Embodiment 17. The composition of embodiment 16, wherein the MYXV is the myxoma virus of any one of embodiments 1-11.

Embodiment 18. The composition of any one of the embodiments 19-21, wherein the PBMCs, BM cells, or a combination thereof comprise autologous cells.

Embodiment 19. The composition of any one of the embodiments 19-21, wherein the PBMCs, BM cells, or a combination thereof are obtained from an allogeneic donor.

Embodiment 20. A method of inhibiting or treating a cancer in a subject in need thereof, comprising administering to the subject a myxoma virus (MYXV) engineered to express an immunomodulatory protein.

Embodiment 21. The method of embodiment 20, wherein the MYXV is the myxoma virus of any one of embodiments 1-11.

Embodiment 22. A method of inhibiting or treating a cancer in a subject in need thereof, comprising administering to the subject a composition of any one of embodiments 12-19.

Embodiment 23. The method of embodiment 22, further comprising adsorbing the myxoma virus ex vivo onto the surface of the PBMCs, BM cells, or a combination thereof.

Embodiment 24. The method of embodiment 23, wherein adsorbing the myxoma virus onto the surface of the PBMCs, BM cells, or a combination thereof comprises exposing the PBMCs, BM cells, or a combination thereof to the myxoma virus under a condition that permit binding of the myxoma virus to the surface of the PBMCs, BM cells, or a combination thereof.

Embodiment 25. The method of any one of the embodiments 22-24, wherein the cancer is a solid tumor.

Embodiment 26. The method of any one of the embodiments 22-25, wherein the cancer has metastasized to a second location in the subject.

Embodiment 27. The method of embodiment 26, wherein the second location comprises a lung, a brain, a liver and/or a lymph node of the subject.

Embodiment 28. The method of any one of the embodiments 22-27, wherein the cancer comprises osteosarcoma, triple negative breast cancer, or melanoma.

Embodiment 29. The method of any one of the embodiments 22-28, further comprising administering to the subject an additional therapeutic agent.

Embodiment 30. The method of embodiment 29, wherein the additional therapeutic agent is administered to the subject prior to administering the MYXV or the composition.

Embodiment 31. The method of embodiment 29 or 30, wherein the additional therapeutic agent is administered to the subject after administering the MYXV or the composition.

Embodiment 32. The method of embodiment 29 or 30, wherein the additional therapeutic agent is administered to the subject as a combination with the MYXV or the composition.

Embodiment 33. The method of any one of the embodiments 22-32, wherein the subject is a human.

Embodiment 34. The method of embodiment 33, wherein the method further comprises selecting a subject that has or is suspected of having a cancer.

Embodiment 35. The method of any one of the embodiments 22-34, wherein the myxoma virus is capable of infecting cells that have a deficient innate anti-viral response.

Embodiment 36. The method of embodiment 35, wherein the cells that have the deficient innate anti-viral response comprise cancer cells.

Embodiment 37. The method of any one of the embodiments 22-36, wherein cells are infected with the myxoma virus expressing an immunomodulatory protein.

Embodiment 38. A kit comprising the myxoma virus of embodiments 1-11 or the pharmaceutical composition of embodiments 12-19.

While this disclosure has been described with an emphasis upon particular embodiments, it will be obvious to those of ordinary skill in the art that variations of the particular embodiments may be used, and it is intended that the disclosure may be practiced otherwise than as specifically described herein. Features, characteristics, compounds, or examples described in conjunction with a particular aspect, embodiment, or example of the invention are to be understood to be applicable to any other aspect, embodiment, or example of the invention. Accordingly, this disclosure includes all modifications encompassed within the spirit and scope of the disclosure as defined by the following claims. We therefore claim as our invention all that comes within the scope and spirit of these claims

We claim:

1. An engineered myxoma virus (MYXV) comprising a transgene encoding one or more immunomodulatory proteins.

2. The engineered MYXV of claim 1, wherein the one or more immunomodulatory proteins is Stimulator of interferon genes (STING), interleukin-12 (IL-12), Fusion-associated small transmembrane (FAST), an immune checkpoint inhibitor, a tumor necrosis factor (TNF) protein, or a combination thereof.

3. The engineered MYXV of claim 2, wherein the IL-12 is an IL-12A.

4. The engineered MYXV of claim 2, wherein the IL-12 is an IL-12B.

5. The engineered MYXV of claim 2, wherein the immune checkpoint inhibitor is a PD-L1 binding molecule.

6. The engineered MYXV of claim 5, wherein the PD-L1 binding molecule is an anti-PD-L1 antibody or an antigen-binding fragment thereof.

7. The engineered MYXV of any one of claims 1-6, wherein the one or more immunomodulatory proteins is capable of stimulating a toll like receptor (TLR), activating Nuclear factor-κB (NF-κB), or activating an interferon regulatory factor (IRF).

8. The engineered MYXV of any one of claims 1-7, wherein the engineered MYXV comprises a modification at or adjacent to one or more genes selected from the group consisting of M001R, M002R, M003.1R, M003.2R, M004.1R, M004R, M005R, M006R, M007R, M008.1R, M008R, M009L, M013, M036L, M063L, M11L, M128L, M131R, M135R, M136R, M141R, M148R, M151R, M152R, M153R, M154L, M156R, M-T2, M-T4, M-T5, M-T7, and SOD.

9. The engineered MYXV of any one of claims 1-8, wherein the engineered MYXV comprises a modification at or adjacent to M011L, M063, M135R, M136R, M-T2, M-T4, M-T5, M-T7 or SOD.

10. The engineered MYXV of claim 8 or claim 9, wherein the modification comprises a deletion or an insertion.

11. The engineered MYXV of any one of claims 8-10, wherein the transgene replaces a portion of M011L, M063, M135R, M136R, M-T2, M-T4, M-T5, M-T7, or SOD.

12. The engineered MYXV of any one of claims 8-11, wherein the transgene is located between the M135R and M136R genes of the genome of the MYXV.

13. The engineered MYXV of any one of claims 8-12, wherein the transgene replaces a portion of M135R.

14. The engineered MYXV of any one of claims 8-13, wherein the engineered MYXV is an M135R knockout.

15. The engineered MYXV of any one of claims 8-11, wherein engineered MYXV is a SOD knockout.

16. The engineered MYXV of any one of claims 1-15, further comprising a reporter gene.

17. The engineered MYXV of claim 15, wherein the reporter gene encodes a fluorescent protein, a luminescent substrate or an enzyme.

18. The engineered MYXV of any one of claims 1-17, wherein the engineered MYXV increases autophagy in infected cells by at least 5% compared to a MYXV that lacks the transgene as determined by an LC3-I to LC3-II conversion assay.

19. The engineered MYXV of any one of claims 1-18, wherein the engineered MYXV increases killing of infected cancer cells by at least 5% compared to a MYXV that lacks the transgene as determined by an in vitro flow cytometric assay.

20. The engineered MYXV of any one of claims 1-19, wherein the engineered MYXV increases killing of uninfected cancer cells by at least 5% compared to a MYXV that lacks the transgene as determined by an in vitro flow cytometric assay.

21. A pharmaceutical composition comprising the engineered MYXV of any one of claims 1-20, and a pharmaceutically acceptable excipient.

22. The pharmaceutical composition of claim 21, wherein the pharmaceutical composition is formulated for systemic administration.

23. The pharmaceutical composition of claim 21, wherein the pharmaceutical composition is formulated for local administration.

24. The pharmaceutical composition of any one of the claims 21-23, wherein the pharmaceutical composition is formulated for parenteral administration.

25. A composition comprising a plurality of cells that is exposed ex vivo to the engineered MYXV of any one of claims 1-20, wherein the plurality of cells comprises a peripheral blood mononuclear cell (PBMC), a bone marrow (BM) cell, or a combination thereof.

26. The composition of claim 25, wherein the plurality of cells is derived from a single subject.

27. A method of inhibiting or treating a cancer in a subject in need thereof, comprising administering to the subject the engineered MYXV of any one of claims 1-20 or the pharmaceutical composition of any one of claims 21-24.

28. A method of inhibiting or treating a cancer in a subject in need thereof, comprising administering to the subject the composition of any one of claims 25-26.

29. The method of claim 28, wherein the engineered MYXV is adsorbed ex vivo onto the surface of at least a portion of the plurality of cells.

30. The method of claim 29, wherein the engineered MYXV is adsorbed by exposing the plurality of cells to the engineered MYXV under a condition that permits binding of the engineered MYXV to a surface of the plurality of cells.

31. The method of claim 28, wherein the engineered MYXV is infected to at least a portion of the plurality of cells.

32. The method of any one of the claims 28-31, wherein the cancer is a solid tumor.

33. The method of any one of the claims 28-31, wherein the cancer has metastasized to a second location in the subject.

34. The method of claim 33, wherein the second location comprises a lung, a brain, a liver and/or a lymph node of the subject.

35. The method of any one of the claims 27-34, wherein the cancer comprises osteosarcoma, triple negative breast cancer, or melanoma.

36. The method of any one of the claims 27-35, further comprising administering to the subject an additional therapeutic agent.

37. The method of claim 36, wherein the additional therapeutic agent is administered to the subject prior to administering the composition.

38. The method of claim 36 or claim 37, wherein the additional therapeutic agent is administered to the subject after administering the composition.

39. The method of any one of claims 36-38, wherein the additional therapeutic agent is co-administered to the subject with the composition.

40. The method of any one of the claims 27-39, wherein the subject is a human.

41. The method of claim 40, further comprising selecting the subject that has or is suspected of having a cancer.

42. The method of any one of the claims 27-41, wherein the engineered MYXV is capable of infecting cells that have a deficient innate anti-viral response.

43. The method of claim 42, wherein cells that have a deficient innate anti-viral response comprise cancer cells.

44. The method of any one of claims 29-43, wherein the plurality of cells is obtained or derived from the subject's tissue.

45. The method of any one of claims 29-43, wherein the plurality of cells are from a donor that is allogeneic to the subject.

46. The method of any one of claims 29-43, wherein the plurality of cells is obtained or derived from a donor's tissue that is HLA-matched, HLA-mismatched, haploidentical, or a combination thereof relative to the subject.

47. A kit comprising the engineered MYXV of any one of claims 1-20, the pharmaceutical composition of any one of claims 21-24, or the composition of any one of claims 25-46.
